(19) United States
(12) Patent Application Publication
(10) Pub. No.: US 2025/0152637 A1
(43) Pub. Date: May 15, 2025

(54) AKKERMANSIA FOR PREVENTING AND/OR TREATING REWARD DYSREGULATION DISORDERS

(71) Applicant: UNIVERSITE CATHOLIQUE DE LOUVAIN, Louvain-la-Neuve (BE)

(72) Inventors: Amandine EVERARD, Dion Valmont (BE); Patrice CANI, Bruxelles (BE); Alice DE WOUTERS D'OPLINTER, Bruxelles (BE); Sabrina HUWART, Zaventem (BE)

(73) Assignee: UNIVERSITE CATHOLIQUE DE LOUVAIN, Louvain-la-Neuve (BE)

(21) Appl. No.: 18/838,757

(22) PCT Filed: Feb. 22, 2023
(86) PCT No.: PCT/EP2023/054478

§ 371 Date: Aug. 15, 2024

(30) Foreign Application Priority Data
Feb. 22, 2022 (EP) 22158054.1

(51) Int. Cl. A61K 35/741; A61K 35/00; A61K 35/742; A61P 25/28

(52) U.S. Cl. CPC A61K 35/741; A61K 35/742; A61P 25/28; A61K 2035/115

(57) ABSTRACT

A composition, which includes one or more bacteria from the genus Akkermansia and/or extracts thereof and/or fragments thereof, for use in the method of preventing and/or treating reward dysregulation disorders. The reward dysregulation disorders include mental disorders, neurological disorders, disorders due to side effects of a treatment and combinations thereof.

### FIELD OF INVENTION

The present invention relates to the field of disorders related to reward dysregulation. In particular, the invention relates to compositions comprising one or more bacteria from the genus Akkermansia and/or extracts and/or fragments thereof for use in preventing and/or treating reward dysregulation disorders.

### BACKGROUND OF INVENTION

Obesity, the prevalence of which has steadily increased in recent decades, is strongly associated with excessive food intake in favor of energy-dense food. This food intake based on hedonic value is controlled by the food reward system. Palatable food-rich in fat and/or sugar-stimulate hedonic, reinforcing and motivational processes of the reward system (Berland C et al, Cell Metab, 2020).

Dopaminergic neurons in the mesocorticolimbic area of the brain are stimulated by palatable food to release dopamine from the ventral tegmental area (VTA) to the striatum (Str) including the nucleus accumbens (Nacc). Long-term overeating is associated with a decrease in the release of dopamine, a decrease in the expression of dopamine receptors proteins (DRD2 and DRD1) and an increase in the expression of the dopamine transporter proteins (DAT) (Volkow N D et al, Philos Trans R Soc Lond B Biol Sci, 2008). This hypofunctioning of the dopamine pathway leads in turn to altered hedonic and motivational behavior feeding (Wang G J et al, Lancet, 2001).

Moreover, food intake is mediated through brain reward systems, which leads to eating disorders when dysregulated (Avena and Bocarsly, Neuropharmacology, 2012; Frank, Curr Psychiatry Rep, 2013). Similarly, drug reward has been shown to overlap circuits of food reward which can lead to addictions in case of dysregulations (Volkow, et al., Curr Topics Behav Neurosci, 2012; Rogers, Pharmacology, Biochemistry and Behavior, 2017).

The gut microbiota is a key regulator in the host metabolism, including in the hypothalamic regulation of food intake through the gut-brain axis (Cani P D et al, Nat Metab, 2019; van de Wouw M et al, J Nutr, 2017). In obesity, the gut microbiota composition is altered and the gut permeability increased. This allows the translocation of bacterial components such as LPS (lipopolysaccharides) across the gut barrier into the systemic circulation, which is called metabolic endotoxemia (Cani P D et al, Diabetes, 2007). LPS can trigger nuclear factor-kappa B (NFkB) and c-Jun N-terminal kinase (JNK) inflammation pathways through TLR4 and induce inflammation in several organs, including the brain, which is associated with a disruption of the blood-brain barrier (Zhao J et al, Sci Rep, 2019).

The causal role of the gut microbiota on dysregulated reward system in the context of obesity was demonstrated recently, more specifically on the reward and hedonic components of the food intake (de Wouters d'Oplinter A et al, Gut Microbes, 2021). One consistent gut microbiota disequilibrium associated with obesity is the decrease abundance of Akkermansia muciniphila (A. muciniphila). Supplementation of Akkermansia muciniphila enables the prevention of body weight gain and metabolic disorders in rodents and humans (Plovier H et al, Nat Med, 2017; Depommier C et al, Nat Med, 2019).

So far, therapy for reward dysregulation disorders mainly account for neuropharmacological compounds and/or psychotherapy. There is therefore a need to provide the state of the art with alternative therapy to treat reward dysregulation disorders.

The present invention is directed towards the treatment and/or prevention of reward dysregulation disorders by taking advantage of the beneficial effects of A. muciniphila on neuronal and behavioral alterations of the reward system.

### SUMMARY

The present invention relates to a composition comprising one or more bacteria from the genus Akkermansia and/or extracts and/or fragments thereof, for use in preventing and/or treating reward dysregulation disorders.

In some embodiments, the bacterium is Akkermansia muciniphila or Akkermansia spp. and combinations thereof.

In some embodiments, the reward dysregulation disorder is selected in a group comprising or consisting of mental disorders, neurological disorders, disorders due to side effects of a treatment and combinations thereof.

In some embodiments, the mental disorder is selected in a group comprising or consisting of addiction-related disorder, eating-related disorder, affective disorders, obsessive compulsive disorders, schizophrenia, attention deficit hyperactivity disorders (ADHD), autism spectrum disorder, anxiety disorder, and the like.

In some embodiments, the addiction-related disorder is selected in a group comprising or consisting of alcohol-related addiction, drug-related addiction, game-related addiction, and the like.

In some embodiments, the neurological disorder is selected in a group comprising or consisting of Parkinson's disease, Tourette Syndrome, and the like.

In some embodiments, the disorder due to side effects of a treatment is selected in a group comprising or consisting of game-addiction, shopping-addiction, eating-addiction such as hyperphagia, hypersexuality, and the like.

In some embodiments, the eating disorder is selected in a group comprising or consisting of bulimia nervosa, binge eating disorder, anorexia nervosa including restricting type and Binge-eating/purging type, pica, rumination disorder, purging disorder, night eating syndrome, avoidant restrictive food intake disorder, overweight-related disorder and obesity-related disorders, food addiction, eating addiction, food craving, food seeking, compulsive eating disorders, impulsive eating disorders, unsuccessful caloric restriction diet, non-responders to weight loss or non-responding to dietary intervention for losing weight, and the like.

In some embodiments, the composition further comprises one or more active agent(s).

In some embodiments, the active agent is a therapeutic agent or a nutritional agent.

In some embodiments, the active agent is a beneficial microbe selected in a group comprising or consisting of bacteria from the family Verrucomicrobia, from the family Tannerellaceae, from the family Clostridiaceae, from the family Peptostreptococcaceae, from the family Prevotellaceae, from the family Methylobacteriaceae, from the genus Parabacteroides, from the genus Turicibacter, from the genus Coprococcus, from the genus Knoellia, from the genus Prevotella, from the genus Staphylococcus, and the like.

In some embodiments, the composition is in the form of a pharmaceutical composition further comprising a pharmaceutically acceptable carrier.

In some embodiments, the composition is in the form of a nutritional composition further comprising a nutritionally acceptable carrier.

The present invention also relates to a composition comprising one or more bacteria from the genus Akkermansia and/or extracts and/or fragments thereof, for use as an adjuvant to a treatment administered to a subject suffering from a reward dysregulation disorder.

In some embodiments, the composition is comprised in a kit, which further comprises means to administer said composition.

### Definitions

In the present invention, the following terms have the following meanings:

“About”, when preceding a value, encompasses plus or minus 10%, or less, of the value of said figure. It is to be understood that the value to which the term “about” refers to is itself also specifically, and preferably, disclosed.

“Adjuvant” refers to a component that potentiates the immune responses to an antigen and/or modulates it towards the desired immune responses. Advantages of adjuvants include the enhancement of the immunogenicity of antigens, modification of the nature of the immune response, the reduction of the antigen amount needed for a successful immunization and an improved immune response in elderly and immunocompromised.

“Bacteria from the genus Akkermansia” refers to bacteria of the phylum Verrucomicrobia, Gram-negative, anaerobic, non-spore forming, non-motile bacteria. Bacteria belonging to the genus Akkermansia may be easily identified by routine procedures, including physiological and biochemical approaches, assessment of their cellular fatty acid profiles, menaquinone profiles and their phylogenetic position, based on 16S rRNA gene sequence analysis based on whole-genome, any tool revealing subspecies-level genetic stratification or putative analysis using CRISPR-Cas loci. Examples of predicted Akkermansia species identified by such techniques are disclosed in Karcher N et al, Genome Biol, 2021.

“Beneficial microbes” refers to microorganisms that may provide health benefits to the hosts, including improvement of the host intestinal microbial balance, maintaining the intestinal gut barrier homeostasis, preventing pathogen colonization, preventing bacterial and viral infections.

“Comprise” is intended to mean “contain”, “encompass” and “include”. In some embodiments, the term “comprise” also encompasses the term “consist of”.

“Eating-related disorders” refers to a particular form of mental disorder, wherein the dysregulation of the reward system is directed towards food intake, in particular palatable food intake. Non-limitative examples of eating-related disorders include anorexia nervosa, bulimia nervosa, binge eating disorder, pica, rumination disorder, purging disorder, night eating syndrome, avoidant restrictive food intake disorder, overweight-related disorder. Eating-related disorders are frequently associated with an abnormal body mass index (BMI), e.g., obesity. In one embodiment, anorexia nervosa includes restricting type and Binge-eating/purging type.

“Enriched composition” refers to a composition in which the population density of bacteria from the genus Akkermansia is enhanced within the total microbial population of the composition.

“Extract” refers to any fraction obtained from the bacteria of interest, or from culture media in which the bacteria of interest were cultured. In practice, extracts include cellular and extracellular extracts. In one embodiment, extracts according to the present invention include metabolites from the bacteria.

“Fragment”, as used herein, refers to any part of the cells of the bacteria of the present invention. Preferably, said fragment is a membrane fraction obtained by a membrane-preparation. Membrane preparations of microorganisms belonging to the genus of Akkermansia can be obtained by methods known in the art. Alternatively, a whole cell preparation is also envisaged. Preferably, the herein described fragment of the microorganism of the present invention retains the capability of preventing and/or treating reward dysregulation disorders.

“Individual” or “subject” refers to an animal individual, preferably a mammalian individual, more preferably a human individual. In some embodiments, an individual may be a mammalian individual. Mammalians include, but are not limited to, all primates (human and non-human), cattle (including cows), horses, pigs, sheep, goats, dogs, cats, and any other mammal which is awaiting the receipt of, or is receiving medical care or was/is/will be the object of a medical procedure, or is monitored for the development of a reward dysregulation disorder. In some embodiments, an individual may be a “patient”, i.e., a warm-blooded animal, more preferably a human, who/which is awaiting the receipt of, or is receiving medical care or was/is/will be the object of a medical procedure, or is monitored for the development of a reward dysregulation disorder. In some embodiments, the individual is an adult (e.g., an individual above the age of 18). In some embodiments, the individual is a child (e.g., an individual below the age of 18). In some embodiments, the individual is a male. In some embodiments, the individual is a female.

“Isolated bacteria” refers to bacteria that are no longer in their natural and/or physiological biotope or habitat. For example, bacteria of interest from a microbiota may be collected and separated from other bacteria and further formulated within a composition. Bacterial separation may be performed according to standard protocols in the field of microbiology, such as, e.g., Gram coloration, antibiotic resistance, ability to grow on specific substrates/culture media, and protocols adapted therefrom.

“Mental disorders” refers to disorders that are characterized by a combination of abnormal thoughts, perceptions, emotions, behavior and relationships with others, as defined by the World Health Organization (WHO). In practice, mental disorders include addiction-related disorder, eating-related disorder, affective disorders, obsessive compulsive disorders, schizophrenia, attention deficit hyperactivity disorders (ADHD), autism spectrum disorder, anxiety disorder, and the like.

“Neurological disorders” refers to disorders that affect the brain, the nerves and the spinal cord. In practice, individuals with neurological disorders may experience symptoms such as, e.g., paralysis, muscle weakness, poor coordination, loss of sensation, seizures, confusion, pain and altered levels of consciousness.

“Pharmaceutically acceptable carrier” refers to a carrier that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the Food and Drug Administration (FDA) in the United States or the European Medicines Agency (EMA) in the European Union.

“Prevention” refers to preventing or avoiding the occurrence of symptom of a reward dysregulation disorder. In the present invention, the term “prevention” may refer to a secondary prevention, i.e., to the prevention of the re-occurrence of a symptom or a relapse of a reward dysregulation disorder.

“Reward dysregulation disorders” refers to disorders wherein the reward system of an individual does not generate a normal, sufficient or adapted response to rewarding stimuli, leading in turn to altered motivational or hedonic induction by the stimuli. Within the scope of the present disclosure, an individual having a reward dysregulation disorder has increased or decreased motivation, and/or increased or decreased pleasure from rewarding stimuli. Typically, reward dysregulation disorders result in an individual's behavioral changes, and promote compulsive behaviors. In practice, reward dysregulation disorders encompass mental disorders and neurological disorders, which are defined below. Diagnosis of individuals with reward dysregulation disorders may be performed by authorized personnel, such as a physician, accordingly to the standards protocols in the field, in particular by monitoring clinical signs, and often with the assistance of a questionnaire.

“Therapeutically effective amount” refers to an amount sufficient to effect beneficial or desired results including clinical results. A therapeutically effective amount can be administered in one or more administrations. In one embodiment, the therapeutically effective amount may depend on the individual to be treated.

“Treating” or “treatment” or “alleviation” refers to both therapeutic treatment and prophylactic or preventative measures, wherein the object is to prevent or slow down (lessen) the targeted reward dysregulation disorder. Those in need of treatment include those already with the reward dysregulation disorder as well as those prone to have the reward dysregulation disorder or those in whom the reward dysregulation disorder is to be prevented. An individual or mammal is successfully “treated” for a reward dysregulation disorder or condition, if, after receiving a therapeutic amount of a composition, pharmaceutical composition, according to the present invention, alone or in combination with another treatment, the patient shows observable and/or measurable reduction in, or absence of, one or more of the symptoms associated with the reward dysregulation disorder; and/or relief to some extent, one or more of the symptoms associated with the reward dysregulation disorder or condition; reduced morbidity and mortality, and improvement in quality of life issues. The above parameters for assessing successful treatment and improvement in the disease are readily measurable by routine procedures familiar to a physician.

Other definitions may appear in context throughout this disclosure.

### DETAILED DESCRIPTION

This invention relates to a composition comprising one or more bacteria from the genus Akkermansia and/or extracts or fragments thereof, for use in preventing and/or treating reward dysregulation disorders.

In some aspects, the invention also relates to the use of a composition comprising one or more bacteria from the genus Akkermansia and/or extracts or fragments thereof, for preventing and/or treating reward dysregulation disorders.

The invention further pertains to the use of a composition comprising one or more bacteria from the genus Akkermansia and/or extracts or fragments thereof, for the preparation or the manufacture of a medicament for preventing and/or treating reward dysregulation disorders.

In another aspect, the invention relates to a method for preventing and/or treating reward dysregulation disorders in an individual in need thereof, comprising the administration of a therapeutically effective amount of a composition comprising one or more bacteria from the genus Akkermansia and/or extracts or fragments thereof.

According to some embodiments, the bacteria from the genus Akkermansia are selected in the group comprising or consisting of Akkermansia muciniphila, Akkermansia glycaniphila, Akkermansia biwaensis, Akkermansia spp, and combinations thereof.

In some embodiments, the bacteria from the genus Akkermansia are Akkermansia muciniphila.

In practice, bacteria belonging to the genus Akkermansia may be identified by any suitable procedures, or a procedure adapted therefrom. In particular, suitable procedures may include physiological and biochemical methods, such as the assessment of the capacity to ferment on selected nutrients, e.g., mannose, raffinose; the assessment of the resistance to some antibiotics; the assessment of specific enzymatic activities, such as, e.g., alpha-galactosidase, beta-galactosidase, alpha-glucuronidase, alkaline phosphatase, L-arginine arylamidase, Leucine glycine arylamidase, Phenylalanine arylamidase; the assessment of their cellular fatty acid profiles, menaquinone profiles; the assessment of their profile by matrix-assisted laser-desorption/ionization time-of-flight mass spectrometry (MALDI-TOF MS); the assessment of their phylogenetic position, based on 16S rRNA gene sequence analysis, based on whole-genome, any tool revealing subspecies-level genetic stratification or putative analysis using CRISPR-Cas loci.

In some embodiments, the bacteria from the genus Akkermansia are isolated. In some embodiments, the bacteria from the genus Akkermansia are isolated from a natural habitat, such as, e.g., the gut microbiota. In practice, the bacteria from the genus Akkermansia may be isolated from feces or caeal content, fresh or frozen, diluted or not in a specific medium (including cryoprotectants and/or antioxidants), accordingly to the standard and ethical procedures in the field.

In practice, bacteria from the genus Akkermansia may be cultured in any suitable culture medium, such as, e.g., the fastidious anaerobe broth (commercially available from DSMZ®, Neogen®), the Pyg Medium (modified) (commercially available from DSMZ®), Columbia Broth (CB), Brain-Heart Infusion (BHI)-Agar medium supplemented with mucus, optionally comprising N-acetylglucosamine and/or N-acetylgalactosamine.

In practice, cultures of bacteria from the genus Akkermansia may be performed at a temperature ranging from about 30° C. to about 42° C., preferably from about 35° C. to about 40° C., more preferably at about 37° C. As used herein, the term “about 30° C. to about 42° C.” includes about 30° C., 31° C., 32° C., 33° C., 34° C., 35° C., 36° C., 37° C., 38° C., 39° C., 40° C., 41° C. and 42° C.

In practice, cultures of bacteria from the genus Akkermansia may be performed in anaerobic conditions, i.e., in the absence of O₂ or partial oxygen depletion.

In some embodiments, the composition of the invention comprises or substantially consists of a microbiota with bacteria from the genus Akkermansia obtained from an individual.

In some embodiments, the composition of the invention is enriched with bacteria from the genus Akkermansia. In one embodiment, the composition of the invention comprises or substantially consist of a microbiota enriched with bacteria from the genus Akkermansia. In one embodiment, the microbiota is a gut microbiota obtained from the feces of an individual. In one embodiment, the microbiota is enriched with bacteria from the genus Akkermansia compared to the microbiota of the individual to be treated.

In practice, bacteria from the genus Akkermansia may be enriched by preferentially stimulating the growth of the bacteria from the genus Akkermansia. For example, enrichment may be performed by modifying physiological conditions of the culture. Examples include, but are not limited to, modification of the composition of the culture media, such as the nutrient composition; and modification of the culture conditions, such as environmental pH value, temperature and oxygen conditions, and the like.

In some embodiments, the bacteria from the genus Akkermansia are isolated and enriched. In some embodiments, the composition of the invention comprises isolated, enriched bacteria from the genus Akkermansia.

In some embodiments, the composition of the invention comprises purified bacteria from the genus Akkermansia. The terms “purified” or “biologically pure” refers to material that is free to varying degrees from components which normally accompany it as found in its native state. “Isolate” denotes a degree of separation from original source or surroundings. “Purify” denotes a degree of separation that is higher than isolation. Thus, a purified isolated bacterium is at least about 90%, 95%, 99% or 100% free of other bacteria, fungi, viruses, or other undefined microbes.

In one embodiment, the bacteria from the genus Akkermansia are viable. As used herein, the term “viable” refers to bacteria that are able to maintain an active metabolism and/or proliferate in a suitable culture medium, under suitable culture conditions, including suitable pH, temperature, salinity, nutrients content, O₂ content.

In one embodiment, the bacteria from the genus Akkermansia are non-viable, wherein said non-viable bacteria retains the capability of preventing and/or treating reward dysregulation disorders. As used herein, the term “non-viable” refers to bacteria that are not able to maintain an active metabolism and/or proliferate in a suitable culture medium, under suitable culture conditions, including suitable pH, temperature, salinity, nutrients content, O₂ content. Example of non-viable bacteria are dormant bacteria, dead bacteria and inactive bacteria.

In one embodiment, the bacteria from the genus Akkermansia are alive. In another embodiment, the bacteria from the genus Akkermansia are dead, wherein said dead bacteria retains the capability of preventing and/or treating reward dysregulation disorders.

In practice, cell viability (active metabolism) may be assessed by measuring the consumption of one nutrient in the culture medium over time. Cell viability (proliferation) may be assessed by spreading a solution containing at least one bacterium of the invention across a petri dish and counting the number of colonies after a determined time of incubation in suitable culture conditions; alternatively, bacteria may be grown in liquid medium, and proliferation may be measured by measuring optical density of the bacterial culture after a determined time of incubation in suitable culture conditions.

In one embodiment, the bacteria from the genus Akkermansia are pasteurized, wherein said pasteurized bacteria retain the capability of preventing and/or treating reward dysregulation disorders. In one embodiment, the pasteurized Akkermansia and/or extracts thereof were heated at a temperature ranging from about 50° C. to about 100° C., preferably from about 60° C. to about 95° C., more preferably from about 70° C. to about 90° C., even more preferably about 70° C.

In some embodiments, the bacteria from the genus Akkermansia are thermally inactivated, wherein said thermally inactivated bacteria retain the capability of preventing and/or treating reward dysregulation disorders. In some other embodiments, the bacteria from the genus Akkermansia are lyophilized, wherein said lyophilized bacteria retain the capability of preventing and/or treating reward dysregulation disorders.

As used herein, the term “extracts” encompasses any components of the bacteria of the invention, in particular it encompasses both cellular and extracellular extracts which retain the capability of preventing and/or treating reward dysregulation disorders.

In practice, cellular extracts include cytoplasmic extracts, membrane extracts, and combination thereof, in particular, extracts obtained from fractionation methods. Cellular extracts may be obtained by any standard chemical (implementing SDS, proteinase K, lysozyme, combinations thereof, and the like) and/or mechanical (sonication, pressure) fractionation approaches, or approaches adapted therefrom.

In practice, extracellular extracts may include the secreted fraction, in particular soluble compounds, or extracellular vesicles (EV). As used herein, the term “extracellular vesicles” encompasses exosomes, exosome-like vesicles, microvesicles (or ectosomes) and apoptotic bodies. In some embodiments, the extracellular extracts are extracellular vesicles. In some embodiments, the extracellular extracts are the secreted fraction. In some embodiments, the extracellular extracts include secreted molecules. In practice, the secreted fraction may be isolated and/or purified from the culture medium, according to any suitable method known in the state of the art, or a method adapted therefrom. Illustratively, the extracellular extracts may be isolated by differential centrifugation from culture medium; by polymer precipitation; by high-performance liquid chromatography (HPLC), combination thereof, and the like.

Non-limitative example of differential centrifugation method from culture medium may include the following steps:
centrifugation for 10-20 min at a speed of about 300×g to about 500×g, so as to remove cells;
centrifugation for 10-20 min at a speed of about 1,500×g to about 3,000×g, so as to remove dead cells;
centrifugation for 20-45 min at a speed of about 7,500×g to about 15,000×g, so as to remove cell debris;
one or more ultracentrifugation for 30-120 min at a speed of about 100,000×g to about 200,000×g, so as to pellet the exosomes.

Alternative methods to isolate exosomes may take advantage of commercial kits, such as, e.g., the exoEasy Maxi Kit (Qiagen®) or the Total Exosome Isolation Kit (Thermo Fisher Scientific®).

In practice, cellular and/or extracellular extracts may comprise nucleic acids, proteins, carbohydrates, lipids and combinations of these such as lipoproteins, glycolipids and glycoproteins, bacterial metabolites, organic acids, inorganic acids, bases, peptides, enzymes and co-enzymes, amino acids, carbohydrates, lipids, glycoproteins, lipoproteins, glycolipids, vitamins, bioactive compounds, metabolites such as metabolites containing an inorganic component, and the like.

As used herein, the term “fragments of the bacteria of the present invention” encompasses any part of the cells of the bacteria of the present invention. Preferably, said fragment is a membrane fraction obtained by a membrane-preparation. Membrane preparations of microorganisms belonging to the genus of Akkermansia can be obtained by methods known in the art. Alternatively, a whole cell preparation is also envisaged. Preferably, the herein described fragment of the microorganism of the present invention retains the capability of preventing and/or treating reward dysregulation disorders.

When used in the context of the present invention, the term “bacteria from the genus Akkermansia of the present invention” also encompasses derivatives or mutants or analogs of said bacteria which retain the capability of preventing and/or treating reward dysregulation disorders.

It is to be understood that the reward dysregulation disorders according to the invention may be diagnosed and/or monitored through the evaluation of clinical signs, with or without the assistance of a dedicated questionnaire. In practice, the diagnosis and/or monitoring of reward dysregulation disorders may be performed by authorized personnel.

According to certain embodiments, the reward dysregulation disorder is selected in a group comprising or consisting of mental disorders, neurological disorders, disorders due to side effects of a treatment and combinations thereof.

In one embodiment, reward dysregulation disorder is a mental disorder.

According to some embodiments, the mental disorder is selected in a group comprising or consisting of addiction-related disorder, eating-disorder, affective disorders, obsessive compulsive disorders, schizophrenia, attention deficit hyperactivity disorders (ADHD), autism spectrum disorder, anxiety disorder, and the like.

In one embodiment, the mental disorder is an addiction-disorder. According to some embodiments, the addiction-related disorder is selected in a group comprising or consisting of alcohol-related addiction, drug-related addiction, tobacco or nicotine addiction, game-related addiction, and the like.

In one embodiment, the mental disorder is an eating-disorder or eating-related disorder. According to certain embodiments, the eating-disorder or eating-related disorder is selected in a group comprising or consisting of bulimia nervosa, binge eating disorder, anorexia nervosa, pica, rumination disorder, purging disorder, night eating syndrome, avoidant restrictive food intake disorder, overweight-related disorder and obesity-related disorders. Within the scope of the present invention, the terms “anorexia nervosa” and “anorexia” are used interchangeably. Within the scope of the present invention, the terms “bulimia nervosa” and “bulimia” are used interchangeably. In one embodiment, anorexia nervosa includes restricting type and Binge-eating/purging type.

According to certain embodiments, the eating-disorder or eating-related disorder is selected in a group comprising or consisting of overweight-related disorders, obesity-related disorders, bulimia, anorexia, pica, rumination disorder, purging disorder, night eating syndrome, avoidant restrictive food intake disorder, food craving, compulsive eating disorders, impulsive eating disorders, unsuccessful caloric restriction diet, non-responders to weight loss or non-responding to dietary intervention for losing weight and the like. According to certain embodiments, the eating-disorder or eating-related disorder is selected in a group comprising or consisting of overweight-related disorders, obesity-related disorders, bulimia, anorexia, pica, rumination disorder, purging disorder, night eating syndrome, and avoidant restrictive food intake disorder. According to certain embodiments, the eating-disorder or eating-related disorder is selected in a group comprising or consisting of overweight-related disorders or obesity-related disorders.

In some embodiments, the eating disorder comprises food addiction, eating addiction, food craving, food seeking, compulsive eating disorders, impulsive eating disorders, unsuccessful caloric restriction diet, non-responders to weight loss or non-responding to dietary intervention for losing weight.

In some embodiments, an eating-disorder or eating-related disorder within the context of the invention is a disorder associated, related, or due to food reward dysregulation or anormal processing. Therefore, in one embodiment, the reward dysregulation disorder of the invention is a food reward dysregulation disorder.

As used herein, an individual with overweight-related disorder has a body mass index (BMI) comprised from about 25.0 to about 29.9. As used herein, an individual with obesity-related disorder has a body mass index (BMI) above about 30.0.

In one embodiment, the individual has a body mass index (BMI) comprised from about 25.0 to about 30.0. In another embodiment, the individual has a body mass index (BMI) above about 30.0.

In one embodiment, the individual has a body mass index (BMI) comprised from about 18.0 to about 25.0. In another embodiment, the individual has a body mass index (BMI) below about 18.0.

In one embodiment, the eating-related disorder is bulimia. In one embodiment, the eating-related disorder is overweight-related disorder or obesity-related disorder. In one embodiment, the eating-related disorder is overweight-related disorder. In one embodiment, the eating-related disorder is obesity-related disorder. In one embodiment, the eating-related disorder is binge eating disorder. In one embodiment, the eating-related disorder is anorexia. In one embodiment, the eating-related disorder is pica. In one embodiment, the eating-related disorder is rumination disorder. In one embodiment, the eating-related disorder is purging disorder. In one embodiment, the eating-related disorder is night eating syndrome. In one embodiment, the eating-related disorder is avoidant restrictive food intake disorder.

In one embodiment, reward dysregulation disorder is a neurological disorder. According to certain embodiments, the neurological disorder is selected in a group comprising or consisting of Parkinson's disease, Tourette Syndrome, and the like.

In one embodiment, reward dysregulation disorder is a disorder due to side effects of a treatment. According to certain embodiments, the disorder due to side effects of a treatment is selected in a group comprising or consisting of game-addiction, shopping-addiction, eating-addiction such as hyperphagia, hypersexuality, and the like.

In some embodiments, the reward dysregulation disorder of the invention is an eating-disorder or an addiction-disorder.

According to some embodiments, the composition is to be administered to an animal individual, preferably a mammalian individual, more preferably a human individual.

In one embodiment, the individual is a mammalian individual. In one embodiment, the individual is a human individual. In one embodiment the individual is a male. In one embodiment, the individual is a female.

According to certain embodiments, the composition is to be administered orally or rectally.

In one embodiment, the composition is administered into the digestive tract. It is to be understood that the digestive tract is the final location of the bacteria according to the invention. In other words, the bacteria according to the invention are intended to be incorporated into the microbiota of the individual.

In one embodiment, the composition is a solid composition. In practice, solid forms adapted to oral administration include, but are not limited to, pill, tablet, capsule, soft gelatin capsule, hard gelatin capsule, dragees, granules, gums, chewing gums, caplet, compressed tablet, cachet, wafer, sugar-coated pill, sugar coated tablet, or dispersing/or disintegrating tablet, powder, solid forms suitable for solution in, or suspension in, liquid prior to oral administration and effervescent tablet.

In one embodiment, the composition is a liquid composition. In practice, liquid form adapted to oral administration include, but are not limited to, solutions, suspensions, drinkable solutions, elixirs, sealed phial, potion, drench, syrup, liquor and sprays.

According to some embodiments, the bacteria are to be administered at a dose comprised from about 1×10² CFU/g to about 1×10¹² CFU/g of the composition, preferably from about 1×10³ CFU/g to about 1×10¹¹ CFU/g of the composition, more preferably from about 1×10⁴ CFU/g to about 1×10¹⁰ CFU/g of the composition. In one embodiment, the bacteria are to be administered at a dose comprised from about 1×10⁴ CFU/g to about 1×10¹¹ CFU/g of the composition, from about 1×10⁵ CFU/g to about 1×10¹¹ CFU/g of the composition, from about 1×10⁶ CFU/g to about 1×10¹¹ CFU/g of the composition, from about 1×10⁷ CFU/g to about 1×10¹¹ CFU/g of the composition or from about 1×10⁸ CFU/g to about 1×10¹¹ CFU/g of the composition.

As used herein, “CFU” stands for “Colony Forming Unit”. As used herein the term “about 1×10² CFU/g to about 1×10¹² CFU/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² CFU/g.

According to some embodiments, the bacteria are to be administered at a dose comprised from about 1×10² cells/g to about 1×10¹² cells/g of the composition. As used herein the term “about 1×10² cells/g to about 1×10¹² cells/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² cells/g.

According to some embodiments, when the composition is a solid composition, the bacteria are to be administered at a dose comprised from about 1×10² CFU/g to about 1×10¹² CFU/g of the composition. As used herein the term “about 1×10² CFU/g to about 1×10¹² CFU/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² CFU/g.

According to some embodiments, when the composition is a solid composition, the bacteria are to be administered at a dose comprised from about 1×10² cells/g to about 1×10¹² cells/g of the composition. As used herein the term “about 1×10² cells/g to about 1×10¹² cells/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² cells/g.

According to some embodiments, when the composition is a liquid composition, the bacteria are to be administered at a dose comprised from about 1×10² CFU/ml to about 1×10¹² CFU/ml of the composition. As used herein the term “about 1×10² CFU/ml to about 1×10¹² CFU/ml” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² CFU/ml.

According to some embodiments, when the composition is a liquid composition, the bacteria are to be administered at a dose comprised from about 1×10² cells/ml to about 1×10¹² cells/ml of the composition. As used herein the term “about 1×10² cells/ml to about 1×10¹² cells/ml” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² cells/ml.

According to certain embodiments, the composition further comprises one or more additional active agent(s).

In one embodiment, the one or more additional active agent(s) is/are a therapeutic agent. In another embodiment, the one or more additional active agent(s) is/are a nutritional agent.

According to certain embodiments, the one or more additional active agent(s) is/are one or more beneficial microbe(s). In other words, in one embodiment, the composition further comprises one or more beneficial microbe(s).

According to some embodiments, the one or more beneficial microbe(s) is/are selected in a group comprising or consisting of bacteria from the family Verrucomicrobia, from the family Clostridiaceae, from the family Peptostreptococcaceae, from the family Prevotellaceae, from the family Methylobacteriaceae, from the family Tannerellaceae such as from the genus Parabacteroides from the genus Turicibacter, from the genus Coprococcus, from the genus Knoellia, from the genus Prevotella, from the genus Staphylococcus, and the like.

According to certain embodiments, the one or more additional active agent(s) is/are one or more therapeutic agent(s) known to prevent and/or treat the reward dysregulation disorder to be treated.

According to certain embodiments, the composition is in the form of a pharmaceutical composition further comprising a pharmaceutically acceptable carrier.

In certain embodiments, pharmaceutically acceptable carriers that may be used in the pharmaceutical compositions according to the invention include, but are not limited to, ion exchangers; alumina; aluminum stearate; lecithin; serum proteins, such as human serum albumin; buffer substances such as phosphates; glycine; sorbic acid; potassium sorbate; partial glyceride mixtures of vegetable oil saturated fatty acids; water; salts or electrolytes, such as protamine sulfate, disodium hydrogen phosphate, potassium hydrogen phosphate, sodium chloride, zinc salts; colloidal silica; magnesium trisilicate, polyvinyl pyrrolidone; cellulose-based substances (e.g., sodium carboxymethyl cellulose), polyethylene glycol; polyacrylates; waxes; polyethylene-polyoxypropylene-block polymers; polyethylene glycol; wool fat; the like; and any combination thereof.

According to certain embodiments, the composition is in the form of a nutritional composition further comprising a nutritionally acceptable carrier.

As used herein, the term “nutritional composition” is intended to refer to any food product, additive food, supplement food, fortified food, including liquid food products and solid food products. In practice, liquid food products include, but are not limited to, soups, soft drinks, sports drinks, energy drinks, fruit juices, lemonades, teas, milk-based drinks, and the like. In practice, solid food products include, but are not limited to candy bars, cereal bars, energy bars, and the like. In one embodiment, the composition of the invention is a food composition.

In some embodiments, the nutritional composition of the invention is for non-therapeutic use, or for use in a non-therapeutic method.

In some aspects, the invention relates to a medicament comprising a therapeutically effective amount of one or more isolated bacteria from the genus Akkermansia and/or extracts and/or fragments thereof, for use in preventing and/or treating reward dysregulation disorders.

The present invention also relates to the use of a composition comprising one or more bacteria from the genus Akkermansia and/or an extract and/or fragments thereof for the manufacture of a medicament for preventing and/or treating reward dysregulation disorders.

In some embodiments, the composition comprising one or more bacteria from the genus Akkermansia and/or an extract and/or fragments thereof is for use as an adjuvant to a treatment administered to a subject suffering from a reward dysregulation disorder.

The present invention also relates to a medical device comprising, consisting of, or consisting essentially of one or more isolated bacteria from the genus Akkermansia and/or extracts and/or fragments thereof, for use in preventing and/or treating reward dysregulation disorders. In one embodiment, the medical device according to the invention comprises a therapeutically effective amount of one or more isolated bacteria from the genus Akkermansia and/or extracts and/or fragments thereof.

According to certain embodiments, the composition is comprised in a kit, which further comprises means to administer said composition.

In some embodiments, the composition, the pharmaceutical composition, the nutritional composition, the medical device or the medicament according to the invention is sterile. In practice, methods for obtaining a sterile pharmaceutical composition include, but are not limited to, GMP synthesis (GMP stands for “Good manufacturing practice”).

The present invention also relates to method of restoring the reward system of a subject, the method comprising administering to said subject a composition comprising one or more bacteria from the genus Akkermansia and/or extracts and/or fragments thereof.

It is understood that the term “reward system”, as used herein, relates to both the hedonic (or ‘liking’) component, and the motivational component of a behavior (e.g., food intake).

In some embodiments, restoring the reward system restores the motivation in the subject. In one embodiment, restoring the reward system enhances or stimulates the motivation in a subject having abnormal decreased motivation. In another embodiment, restoring the reward system decreases the motivation in a subject having abnormal increased motivation.

The present invention also relates to method of modulating the dopaminergic system of the central nervous system in a subject in need thereof, the method comprising administering to said subject a composition comprising one or more bacteria from the genus Akkermansia and/or extracts and/or fragments thereof in an amount effective to induce the reward response. In some embodiments, the reward response simulates a desired state of being in the subject.

The present invention also relates to method of modulating the opioid system in a subject in need thereof, the method comprising administering to said subject a composition comprising one or more bacteria from the genus Akkermansia and/or extracts and/or fragments thereof in an amount effective to induce the reward response. Indeed, the modulation of the opioid system has an impact on the ‘liking’ component. In some embodiments, the reward response simulates a desired state of being in the subject. In a particular embodiment, without being bound by any theory, the effect of one or more bacteria from the genus Akkermansia and/or extracts and/or fragments thereof on the reward response modulates the opioid system.

The present invention also relates to method of reducing the expression of lipoprotein lipase (LPL) in the striatum a subject in need thereof, the method comprising administering to said subject a composition comprising one or more bacteria from the genus Akkermansia and/or extracts and/or fragments thereof in an amount effective.

The present invention also relates to method of reducing brain inflammation of a subject in need thereof, the method comprising administering to said subject a composition comprising one or more bacteria from the genus Akkermansia and/or extracts and/or fragments thereof in an amount effective.

The present invention also relates to method of reducing the release of proinflammatory factors, preferably proinflammatory cytokines, and/or increasing the release of anti-inflammatory factors, preferably proinflammatory cytokines, of a subject in need thereof, the method comprising administering to said subject a composition comprising one or more bacteria from the genus Akkermansia and/or extracts and/or fragments thereof in an amount effective.

The present invention also relates to method of improving the efficacy of a reward dysregulation disorder treatment, the method comprising administering to said subject a composition comprising one or more bacteria from the genus Akkermansia and/or extracts and/or fragments thereof, before, concomitantly or after the administration of the reward dysregulation disorder treatment.

Non-limitative examples of reward dysregulation disorder treatment include medications against addiction (e.g., methadone for opioids addiction, disulfiram for alcohol addiction, or phentermine-topiramate for bulimia), behavioral or psychological counseling.

The present invention further relates to a method for restoring the reward function in an individual in need thereof. In one embodiment, the method comprises the administration of a composition comprising one or more active ingredients or substances that increase the level of bacteria from the genus Akkermansia in the microbiota of the subject. In some embodiments, the composition is a prebiotic. In a particular embodiment, the method comprises the administration of a composition comprising one or more bacteria from the genus Akkermansia and/or extracts and/or fragments thereof. In one embodiment, this method is non-therapeutic.

The present invention also relates to a method for reducing the reward eating in an individual in need thereof. In one embodiment, the method comprises the administration of a composition comprising one or more active ingredients or substances that increase the level of bacteria from the genus Akkermansia in the microbiota. In a particular embodiment, the method comprises the administration of a composition comprising one or more bacteria from the genus Akkermansia and/or extracts and/or fragments thereof. In one embodiment, this method is non-therapeutic.

The present invention further relates to a method for reducing the intake of palatable diet in an individual in need. In one embodiment, the method comprises the administration of a composition comprising one or more active ingredients or substances that increase the level of Akkermansia in the microbiota. In a particular embodiment, the method comprises the administration of a composition comprising one or more bacteria from the genus Akkermansia and/or extracts and/or fragments thereof. In one embodiment, this method is non-therapeutic.

The present invention further relates to a method for restoring the motivation to eat in an individual in need. In one embodiment, restoring the motivation means enhancing or stimulating motivation in a subject having abnormal decreased motivation. In another embodiment, restoring the motivation means decreasing motivation in a subject having abnormal increased motivation. In one embodiment, the method comprises the administration of a composition comprising one or more active ingredients or substances that increase the level of Akkermansia in the microbiota. In a particular embodiment, the method comprises the administration of a composition comprising one or more bacteria from the genus Akkermansia and/or extracts and/or fragments thereof. In one embodiment, this method is non-therapeutic.

The present invention further relates to a method for restoring appetite in an individual in need. In one embodiment, restoring appetite means enhancing or improving appetite. In another embodiment, restoring appetite means decreasing or inhibiting appetite. In one embodiment, the method comprises the administration of a composition comprising one or more active ingredients or substances that increase the level of Akkermansia in the microbiota. In a particular embodiment, the method comprises the administration of a composition comprising one or more bacteria from the genus Akkermansia and/or extracts and/or fragments thereof. In one embodiment, this method is non-therapeutic.

The present invention further relates to a method for enhancing ‘liking’ reaction in an individual in need. In one embodiment, the method comprises the administration of a composition comprising one or more bacteria from the genus Akkermansia and/or extracts and/or fragments thereof, in particular a composition comprising pasteurized Akkermansia. In one embodiment, the method comprises the administration of a composition comprising one or more active ingredients or substances that increase the level of Akkermansia in the microbiota. In one embodiment, this method is non-therapeutic.

### BRIEF DESCRIPTION OF THE DRAWINGS

FIGS. 1A-1D are a set of histograms and graph showing that obesity is associated with an alteration of the food reward system. (A) Food preference test showing HFHS and CT intake in grams after 3 hours of test by lean and DIO mice. (B-C) Operant conditioning test showing the number of active lever press during the four progressive ratio sessions (PR) and the breaking point during the PR4 by lean and DIO mice. (D) Nucleus accumbens mRNA relative expression of dopamine receptor 2 (DRD2), dopamine receptor 1 (DRD1), tyrosine hydroxylase (TH) and dopamine transporter (DAT) measured by real-time qPCR in lean and DIO mice. Data are shown as mean+−SEM. P-value were obtained after Two-way ANOVA followed by Bonferroni post-hoc test (n=12 CT/10 DIO) (A) after two-way ANOVA repeated measure followed by Bonferroni post-hoc test (n=12/group) (B-C) after unpaired Student's t-test or non-parametric Mann-Whitney test (n=Sep. 10, 2012/group) (B, C, D) *: p-value<0.05; ***: p-value<0.001; ****: p-value<0.0001 between lean vs DIO. $$$$: p-value<0.0001 between CT vs HFHS food intake.

FIGS. 2A-2F are a set of histograms showing that obesity is associated with inflammation and blood-brain barrier alterations in reward-related brain areas. (A) Nucleus accumbens mRNA relative expression of ionized calcium-binding adapter (Iba1), glial fibrillary acidic protein (Gfap), cluster of differenciation 45 (Cd45), interleukin 1 beta (Il1b), tumor necrosis factor alpha (Tnfa) and interleukin 6 (IL6) measured by real-time qPCR in lean (black) and DIO (grey) mice. (n=10/group). (B-C) Representative immunofluorescence of the dorsal striatum (D. Str), the ventral striatum (V. Str) total, core and shell and quantification of both the area occupied by astrocytes cells (C) and the GFAP+ cells in these regions (B) of lean (black) and DIO (grey) mice (n=4-5/group). (D-E) Representative immunofluorescence of the dorsal striatum (D. Str), the ventral striatum (V. Str) total, core and shell and quantification of both the area occupied by microglial cells € and the Iba1+ cells in these regions (D) of lean (black) and DIO (grey) mice (n=5/group). (F) Ventral striatal mRNA relative expression of claudin-1 (Cldn1), claudin-5 (Cldn5), zonula occludens 1 (Zol) and occludin (Ocln) measured by real-time qPCR in lean (black) and DIO (grey) mice (n=10/group). Data are shown as mean+−SEM. P-value were obtained after unpaired Student's t-test or non-parametric Mann-Whitney test. *: p-value<0.05; **: p-value<0.01; ***: p-value<0.001 between lean vs DIO.

FIGS. 3A-3D are a set of histograms showing details of immunofluorescence quantification. (A-B) Immunofluorescence quantification of the area occupied by astrocytes cells (A) and the Gfap+ cells (B) in the right and left dorsal striatum (D. Str), total ventral striatum (V. Str total), ventral striatum core (V. Str core) and ventral striatum shell (V. Str shell) of lean (black) and DIO (grey) mice (n=4-5/group). (C-D) Immunofluorescence quantification of the area occupied by microglial cells (C) and the Iba1+ cells (D) in the right and left dorsal striatum (D. Str), total ventral striatum (V. Str total), ventral striatum core (V. Str core) and ventral striatum shell (V. Str shell) of lean (black) and DIO (grey) mice (n=5/group). Data are shown as mean+SEM. P-values were obtained after unpaired Student's t-test or non-parametric Mann-Whitney test. *: p-value<0.05; **: p-value<0.01 between lean vs DIO.

FIGS. 4A-4B are a set of histograms and graph showing that Akkermansia muciniphila administration restores the motivational component of food reward associated with obesity. Operant conditionning test showing the number of active lever press during the four progressive ratio sessions (PR) and the breaking point during the PR4 by DIO mice treated with placebo (DIO_placebo) and A. muciniphila (DIO_Akk) (A) and the maximal number of pellets earned (B). Data are shown as mean+−SEM. P-value were obtained after two-way ANOVA repeated measure followed by Bonferroni post-hoc test (n=6/group); or after unpaired Student's t-test or non-parametric Mann-Whitney test (n=6/group). *: p-value<0.05; **: p-value<0.01 between DIO_placebo vs DIO_Akk. $: p-value<0.05 between CT vs HFHS food intake.

FIGS. 5A-5C are a set of histograms showing that Akkermansia muciniphila administration reduces markers of systemic and in reward-related area inflammation associated with obesity as well as LPL striatal expression. (A) Striatal mRNA relative expression of ionized calcium-binding adapter (IBA-1), toll-like receptor 4 (TLR4), glial fibrillary acidic protein (GFAP), and cluster of differentiation 45 (CD45) measured by real-time qPCR in DIO mice treated with placebo (DIO_placebo) and A. muciniphila (DIO_Akk) (n=8-10/group). (B) Plasma concentration of the cytokines tumor necrosis factor alpha (TNFα). (C) Striatal mRNA relative expression of lipoprotein lipase (LPL) measured by real-time qPCR in DIO mice treated with placebo (DIO_placebo) andA. muciniphila (DIO_Akk) (n=8-10/group). Data are shown as mean+−SEM. P-value were obtained after unpaired Student's t-test or non-parametric Mann-Whitney test. *: p-value<0.05; **: p-value<0.01 between DIO_placebo vs DIO_Akk.

FIGS. 6A-6B are a set of histograms and showing that pasteurized Akkermansia muciniphila administration restores the liking component of food reward associated with obesity. Food preference test showing HFHS and CT intake in grams (A) and preference for HFHS in percentage after 3 h of test (B) by DIO mice treated with placebo (DIO_Placebo) and pasteurized A. muciniphila (DIO_Akkpast). The percentage of food preference was calculated based on HFHS intake (g) during the food preference test divided by the total food intake (g) eaten during the food preference test. Data are shown as mean+SEM. p-values were obtained after two-way ANOVA followed by Bonferroni post-hoc test. (n=7-8/group) **: p-value<0.01 between DIO_Placebo and DIO_Akkpast HFHS intake. $$$$: p-value<0.0001 between CT vs. HFHS food intake (A). p-values were obtained after Student t-test. (n=7-8/group) ****: p-value<0.0001 between DIO_Placebo and DIO_Akkpast (B).

FIGS. 7A-7C are a set of graphs showing that pasteurized Akkermansia muciniphila administration improves the wanting component of food reward associated with obesity. Operant conditioning test showing the number of active lever press during the four progressive ratio sessions (PR) (A), the number of active lever press during the four progressive ratio sessions (PR) (B) and the maximal number of pellets earned (C) by DIO mice treated with placebo (DIO_placebo) and pasteurized A. muciniphila (DIO_Akkpast). Data are shown as mean+−SEM. p-value were obtained after two-way ANOVA repeated measure followed by Bonferroni post-hoc test (n=6-7/group) (A) or after Mann-Whitney test (n=6-8/group) (B, C). *: p-value<0.05 between DIO_Placebo and DIO_Akkpast.

FIG. 8 is a histogram showing that pasteurized Akkermansia muciniphila administration does not impact the expression of inflammatory markers in the striatum. Striatal mRNA relative expression of ionized calcium-binding adapter (Iba1), glial fibrillary acidic protein (Gfap), interleukin 6 (Il6), interleukin 1 beta (Il1b), tumor necrosis factor alpha (Tnfa) and toll-like receptor 4 (Tlr4) measured by real-time qPCR in DIO mice treated with placebo (DIO_Placebo) and pasteurized A. muciniphila (DIO_Akkpast). Data are shown as mean+−SEM. p-value were obtained after Student t-test or Mann-Whitney test (n=7-8/group).

FIGS. 9A-B are sets of histograms showing that pasteurized Akkermansia muciniphila administration increases the area occupied by microglia cells in the striatum. Representative immunofluorescence of the dorsal striatum (D. Str), the ventral striatum (V. Str) total, core and shell and quantification of the Iba1+ cell count (A) and the area occupied by microglial cells (B) in these regions of DIO_Placebo and DIO_AkkPast mice (n=4-6/group). Data are shown as mean+−SEM. p-value were obtained after Student t-test between DIO_Placebo and DIO_Akkpast. *: p-value<0.05 between DIO_Placebo and DIO_Akkpast.

FIG. 10 is a histogram showing that pasteurized Akkermansia muciniphila administration modulates the expression of markers of M2 anti-inflammatory phenotypes of microglial cells in the striatum. Striatal mRNA relative expression of cluster of differentiation 11b (Cd11b), cluster of differentiation 206 (Cd206) and arginase 1 (Arg1) measured by real-time qPCR in DIO mice treated with placebo (DIO_Placebo) and pasteurized A. muciniphila (DIO_Akkpast). Data are shown as mean+−SEM. p-value were obtained after Student t-test or Mann-Whitney test (n=7-8/group).

FIG. 11 is a histogram showing that pasteurized Akkermansia muciniphila administration reduces the expression of striatal LPL. Striatal mRNA relative expression of lipoprotein lipase (Lpl) measured by real-time qPCR in DIO mice treated with placebo (DIO_Placebo) and pasteurized A. muciniphila (DIO_Akkpast). Data are shown as mean+−SEM. p-value were obtained after Student t-test (n=7-8/group).

### EXAMPLES

The present invention is further illustrated by the following examples.

### Example 1: Diet-Induced Obese Mice Show Behavioral and Neuronal Alterations in Response to Food Reward

### Materials and Methods

Mice and experimental design: All mouse experiments were approved by the ethical committee for animal care of the Health Sector of the UCLouvain, Université catholique de Louvain under the specific number 2017/UCL/MD/005 and performed in accordance with the guidelines of the local ethics committee and in accordance with the Belgian Law of May 29, 2013 regarding the protection of laboratory animals. Body weight, food and water intake were recorded once a week. Body composition was assessed by using 7.5 MHz time domain-nuclear magnetic resonance (TD-NMR, LF50 Minispec, Bruker, Rheinstetten, Germany). A cohort of 9-week-old specific-opportunistic and pathogen-free (SOPF) male C57BL/6J mice (Janvier laboratories, France) were housed in a controlled environment (room temperature of 22±2° C., 12 h daylight cycle) in groups of two mice per cage, with free access to sterile food (irradiated) and sterile water. Upon delivery, mice underwent an acclimatization period of one week, during which they were fed a control diet (CT, AlN93Mi, Research Diet, New Brunswick, NJ, USA). Mice were then randomly divided in two groups (40 mice, n=20/group), and fed for 8 weeks with control low-fat diet (CT, AlN93Mi) or a high-fat diet (HFD, 60% fat and 20% carbohydrates (kcal/100 g) D12492i, Research diet, New Brunswick, NJ, USA). After 4 weeks of follow-up, the mice entered the behavioral cages to perform the food preference test and the operant wall test. During this last test, mice were food-restricted and body weights were maintained at 85% of the initial body weight (before the behavioral tests), as previously described (de Wouters d'Oplinter A et al, Gut Microbes, 2021). The caloric restriction allowed to potentiate the reward response to the stimuli.

Food preference test: During 3 hours in the daylight, mice were exposed to two diets: a low-fat, control diet (CT, AlN93Mi, Research diet, New Brunswick, NJ, USA) or a high-fat high-sucrose diet (HFHS, 45% fat and 27.8% sucrose (kcal/100 g) D17110301i, Research diet, New Brunswick, NJ, USA) in Phenotyper chambers (Noldus, The Netherlands). The food intakes were recorded during a 3-hour session in the end of the light phase, in satiated state (access to food ad libitum before and after the test).

Operant wall test: The wanting component is linked to the motivation to obtain a reward and is evaluated by an operant wall test as previously described with some adaptations (Cansell C et al, Molecular psychiatry, 2014). Each session of the test was conducted during the end of the light phase, in operant conditioning chambers (Phenotyper chambers, Noldus, The Netherlands) and analyzed by the provided software (Ethovision XT 14). The mice had intermittent access to an operant wall in their home cages. The operant wall system is composed of two levers and two lights and a pellet dispenser. One lever is arbitrarily designated as active, meaning that pressing on this lever initiates the delivery of a sucrose pellet (5-TUT peanut butter flavored sucrose pellet, TestDiet, St. Louis, MO) and is associated with a light on. On the other side, another lever associated with a light off, is arbitrarily designated as inactive and will never deliver a reward. Mice were trained for the system twice overnight on a fixed-ration schedule (one lever press corresponds to one reward), then underwent 2 sessions of 2 hours. Mice were then shifted to progressive ratio sessions (1 h 30), the number of lever press to obtain a reward is incrementally increased (n+3) for every pellet.

Tissue sampling: At the end of each experiment, mice were maintained under caloric restriction and exposed for 1 h to HFHS before anesthesia with isoflurane (Forene, Abbott, England). This aims to mimic the conditions of the behavioral tests and stimulate the reward system. Then the mice were anesthetized by isoflurane and euthanatized by exsanguination and cervical dislocation. Blood was sampled from the portal and cava veins. Striatum and nucleus accumbens were precisely dissected and immediately immersed into liquid nitrogen, then stored at −80° C. for further analysis.

RNA preparation and real-time qPCR analysis: Total RNA was extracted from the striatum using TriPure reagent (Roche). cDNA was prepared by reverse transcription of 1 μg total RNA using the GoScript Reverse Transcriptase kit (Promega, Madison, WI, USA). Real-time PCR was performed with the QuantStudio 3 real-time PCR system (Thermo Fisher, Waltham, MA, USA). Rpl19 RNA was chosen as the housekeeping gene. All samples were performed in duplicate, and data were analyzed according to the 2-ΔΔCT method. The identity and purity of the amplified product were assessed by melting curve analysis at the end of amplification.

Statistical analysis: Statistical analyses were performed using GraphPad Prism version 9.1.2 for Windows (GraphPad Software, San Diego, CA, USA). Data are expressed as mean±SEM. Differences between two groups were assessed using unpaired Student's t-test. In case variance differed significantly between groups according to the Fisher test, a non-parametric (Mann-Whitney) test was performed. Difference between two groups and different time points was assessed using a two-way ANOVA repeated measurement, followed by Bonferroni post-hoc test.

### Results

9-week-old lean or diet-induced obese mice were followed and fed with control, low-fat diet (CT, Lean group) or a high-fat diet (HFD) for 9 weeks. After 4 weeks of feeding the respective diet to establish obesity, the liking and the wanting components of the food reward system were assessed with behavioral tests.

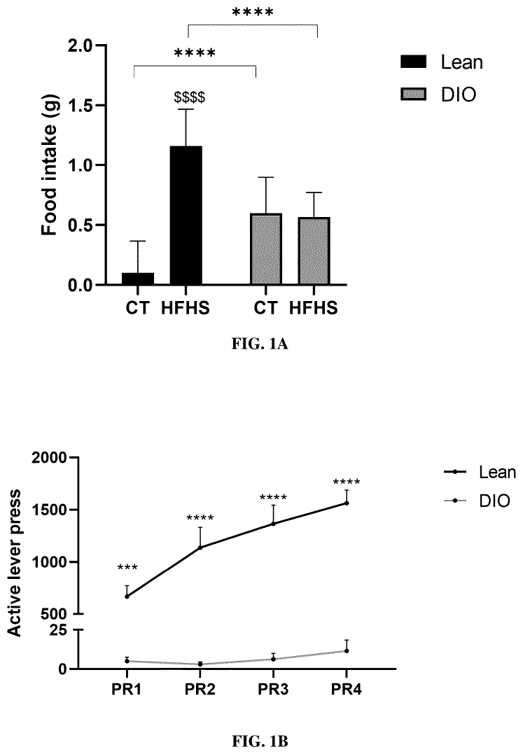

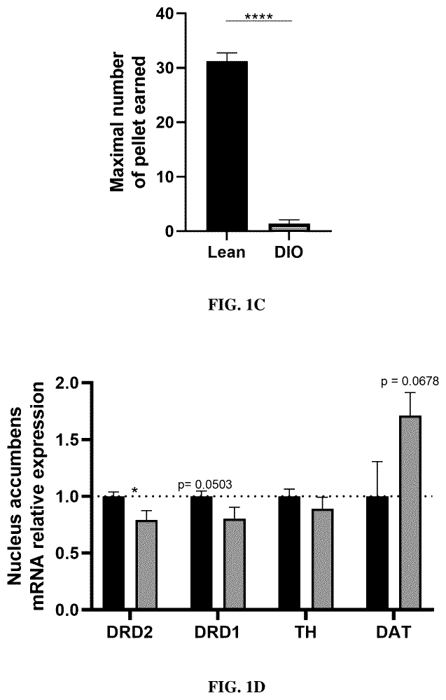

The liking or hedonic component of food intake was first assessed by analyzing the tropism towards palatable food (HFHS, high-fat high-sucrose) over control food (CT) during a food preference test. Mice were exposed to both diets during a 3-hour session at the end of the light phase, in a satiated state (access to food ad libitum before and after the test). In this context, an HFHS preference would suggest a rewarding response specifically to palatable food. As expected, and consistent with the literature (Delbes A S et al., Front Endocrinol, 2018; Carlin J et al., Obesity, 2013), lean mice showed a greater preference for palatable diet (HFHS) than the control diet as shown by the higher intake of HFHS compared to CT diet intake (p<0.0001 between CT vs HFHS in Lean group, FIG. 1A); in contrast, obese mice (DIO) had no preference for HFHS over the CT diet (p=0.77 between CT vs. HFHS in the DIO group, FIG. 1A). Importantly, DIO mice ate more than 2-fold less HFHS intake than lean mice (p<0.0001 between Lean and DIO mice, 1.16 g vs. 0.57 g, respectively). As this test represents the first exposure to palatable diet, able to stimulate the mesocorticolimbic system and induce pleasure, the absence of preference for palatable food and the reduced intake of this diet in obese mice suggests a dysregulation of the hedonic component of food intake associated with obesity.

Then, the motivational component of the food reward was assessed by subjecting the mice to an operant conditioning task in which their eagerness to obtain rewarding food (peanut butter-flavoured sucrose pellets) was tested. Lean and DIO mice were first evaluated for incentive motivation on a fixed-ratio schedule: one press on the lever delivered one sucrose pellet. After 4 sessions using a fixed ratio (FR), mice were shifted to progressive ratio sessions (PR), which requires an increasing number of lever presses to obtain a new sucrose pellet [3 lever presses more for each subsequent reinforcer (r=3n+3; n=reinforcer number)]. The PR sessions thereby measure the amount of effort an animal was willing to exert to obtain food rewards and relies on the motivational aspect of the reward system. Compared to lean mice, DIO mice pressed significantly less on the lever to obtain a reward during PR sessions, suggesting a reduction of motivation (p<0.0001 between Lean and DIO mice, FIG. 1B-C). Consistent with the number of lever presses, the breaking-point or the maximum number of sucrose pellets earned during a session was significantly lower in the DIO group than the the lean group (p=0.0001 and p<0.0001 between Lean and DIO mice in PR1 and PR2-4, FIG. 1B-C). These results show the dysregulation of the motivational component of the reward system associated with obesity, as previously described in the literature (Delbes A S et al., Front Endocrinol, 2018).

Following the observation of the dysregulation of the reward system from a behavioral perspective in obese animals compared to lean mice, the reward system was further investigated by analyzing the dopaminergic system in mesocorticolimbic structures of the brain (FIG. 1D). The expressions of dopaminergic receptors 2 (Drd2) and 1 (Drd1) were decreased in the ventral striatum of obese mice (p=0.028 and p=0.050, respectively) whereas the dopamine transporter (Dat), which is responsible for the recapture of dopamine, tended to increase (p=0.068; FIG. 1D). The expression of tyrosine hydroxylase (Th), the rate-limiting enzyme synthetizing dopamine, was not affected in DIO mice.

The changes in the expression of key dopaminergic markers associated with obesity, show a downregulation of the dopamine pathway and reflect the behavioral dysregulations of liking and wanting components of food reward observed during the food preference and the operant wall tests, respectively. Altogether, these results show alterations of the reward system in obese mice, on behavioral and neuronal approaches, as previously described in the literature (de Wouters d'Oplinter A et al., Gut Microbes, 2021).

### Example 2: Obesity is Associated with Inflammation and Blood-Brain Barrier Alterations in Reward-Related Brain Areas

### Materials and Methods

Mice experiment, tissue sampling, RNA preparation, real-time qPCR and statistical analysis: see example 1.

Immunofluorescence: At the end of the experiment (see example 1), mice were anesthetized by isoflurane and transcardiacly perfused using a solution of cold phosphate-buffered saline (PBS) and then a solution of cold 4% (w/v) paraformaldehyde (PFA). The entire brain was carefully harvested, post-fixed in 4% PFA overnight at 4° C., cryoprotected overnight at 4° C. in a solution of sucrose 30% (w/v), subsequently frozen in cold iso-pentane and stored at −80° C., as previously described (Everard A et al. Nat Commun 2019). Twenty micrometers thick serial coronal cryosection from fixed brain were mounted on SuperFrost Plus slides (Menzel Gläser) and kept at −20° C. For Nucleus accumbens and striatum, 8 serial sections per animal were harvested from bregma 0.61 mm to 1.41 mm according to The Mouse Brain in stereotaxic coordinates (Paxinos, Franklin). Immunofluorescence was performed using Tyramide-signal amplification (TSA) technology, as previously described (Everard A et al., Nat Commun 2019). Briefly, after antigen retrieval (Dako S1699) by heating (2100 Antigen Retriever, from Aptum) the endogenous peroxidases were inhibited in a solution of MeOH with H₂O₂ 0.1% (v/v). Then the sections were incubated for 45 min in blocking solution (TBS, BSA 5%, Tween 20 0.1%) and then incubated overnight with a primary antibody (anti-GFAP 1/10 000, ab5804 from Merck or anti-Iba-1 1/500, PA5-27436 from ThermoFischer). After washing, sections were incubated for 1 h with Horse Radish Peroxidase-conjugated secondary antibody (DAKO K4003). The fluorescent signal was amplified using Alexa Fluor 488 Tyramide Reagent (B40953 from ThermoFischer). Finally, nuclei were stained with Hoechst 33342 (H1399 Invitrogen). Slides were dehydrated and mounted with Dako Fluorescence Mounting Medium. Fluorescent GFAP scans were obtained using Oyster scanner (3DHistech Panoramic P250 Flash III) and fluorescent Iba-1 scans using Zeiss scanner (Axioscan.z1). After blinding procedure, using Fiji software48 the region of interest (ROI) corresponding to the nucleus accumbens core, shell and striatum dorsal were delimited on each section using The Mouse Brain in stereotaxic coordinates (Paxinos, Franklin) as reference and % of green area were measured. Positive neurons were manually counted within each ROI and a mean value was obtained for each animal. At least, three brain sections per animal were considered.

### Results

Obesity has been extensively described as being associated with low-grade inflammation in several tissues including the brain (Guillemot-Legris O et al., Trends Neurosci, 2017). Importantly, the so-called neuroinflammation has been reported to alter brain functions (Decarie-Spain L et al., Brain Behav Immun Health, 2021). Therefore, it is hypothesized that the dysregulation of dopaminergic pathways during obesity could be linked to inflammation in reward-related brain areas. Since inflammation in the brain is associated with the activation of microglia and astrocytes, as reflected by an increase in the expression of ionized calcium-binding adaptor protein-1 (Iba1) and glial fibrillary acidic protein (GFAP), respectively, the expression of the receptor for LPS (Tlr4), infiltrating immune cell markers (cluster of differentiation 45 (Cd45)) and proinflammatory cytokines (interleukin-6 (Il6), interleukin-1 β (Il1b) and tumor necrosis factor α were analyzed

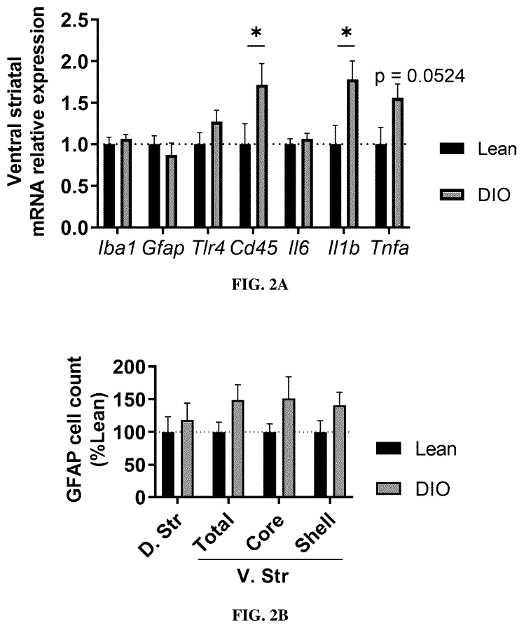

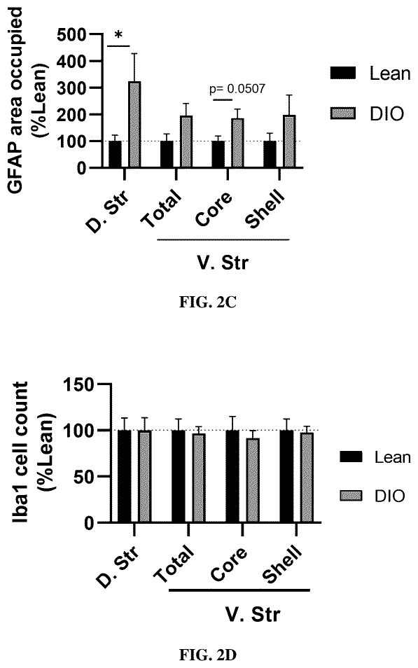

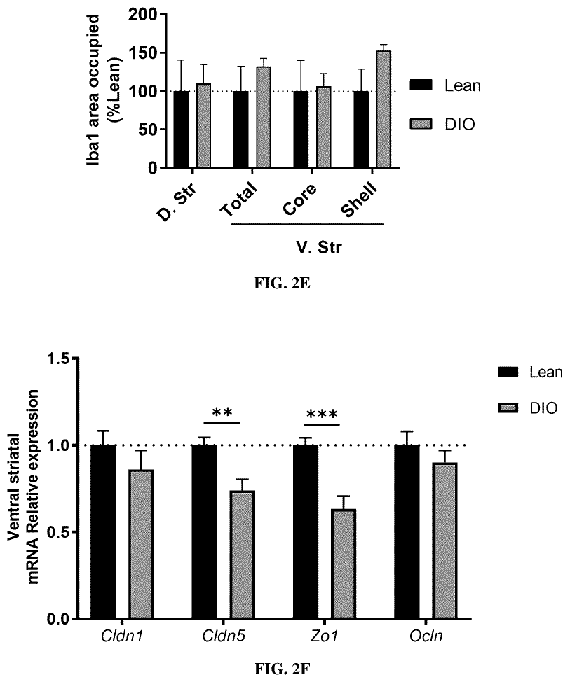

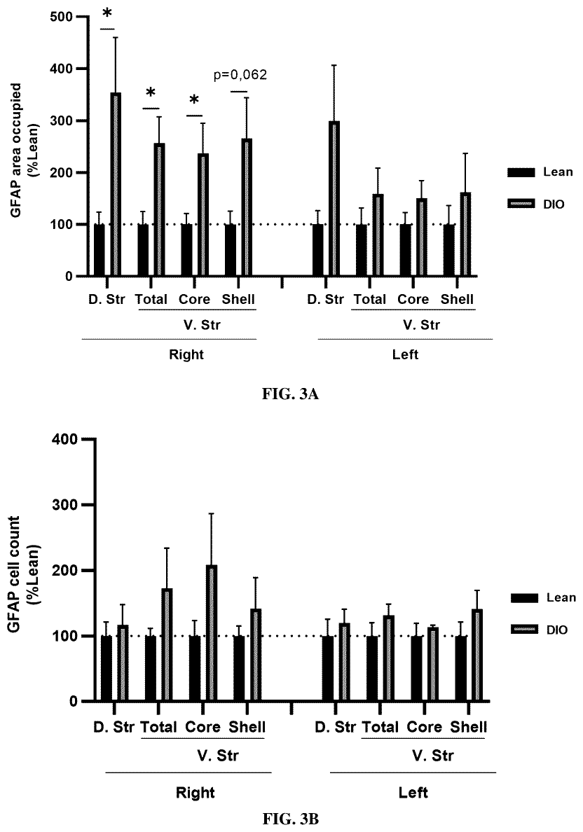

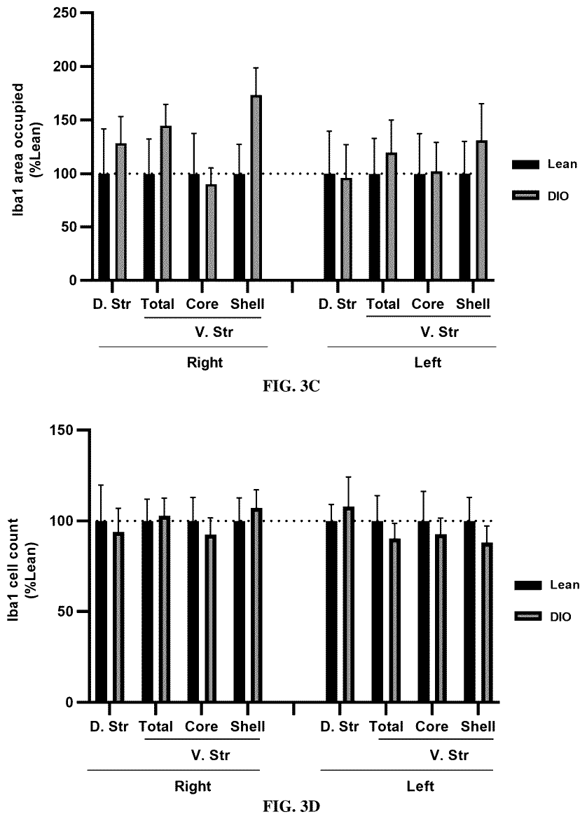

Significantly increased expressions of CD45, IL-1B (p=0.044 and p=0.025 respectively) and a trend for an increased expression of TNFα (p=0.0524) were detected in obese mice compared to lean mice (FIG. 2A), suggesting the induction of neuroinflammation in the mesocorticolimbic area by high-fat-diet-induced obesity. Immunohistochemichal staining for GFAP and Iba1 in the striatum was performed in reward-related area. DIO induced astrocyte activation in the dorsal striatum and ventral striatum core, as shown by the increase staining of—labelled area in the DIO group compared to the lean group (p=0.049 and 0.051, respectively, for the comparison between Lean and DIO mice, FIG. 2B-C). Surprisingly, astrocytes were mainly activated in the right dorsal and the ventral striatum, while these regions in the left hemisphere did not present astrocyte activation (FIG. 3A-D). Iba1 immunostaining did not show a difference in microglial cell activation (FIG. 2D-E).

The blood-brain barrier (BBB) is essential to protect brain structures from toxins, pathogens and excess immune cell infiltration and to maintain neuronal integrity. Since neuro-inflammation is associated with disruption of the BBB in several neurological disorders including some associated with obesity, the integrity of the BBB in reward-related brain area was further analyzed by measuring expression of key tight junction proteins. Interestingly, the expression levels of claudin-5 (Cldn5) and zonula occludens 1 (Zol) were significantly decreased in obese mice compared to lean mice (p=0.0033 and p=0.0004 for the comparison between Lean and DIO mice) whereas claudin-1 (Cldn1) and occludin (Ocln) levels were not changed (FIG. 2F).

This line of results showed for the first time that the behavioral and neuronal dysfunctions of the reward system in obesity are associated with markers of neuroinflammation and BBB alterations.

### Example 3: The Administration of Akkermansia muciniphila Restores the Motivational Component of Food Reward that is Altered by DIO

The gut microbiota plays a key role in systemic inflammation during obesity. Indeed, a chronic high-fat diet is associated with alterations of the gut microbiota composition, as well as with an increase in the translocation of the bacterial components lipopolysaccharides (LPS), through the gut barrier. This increase in plasma LPS, also called metabolic endotoxemia, generates low-grade inflammation by the activation ofTLR4. Saturated fatty acids also activate TLR4 and potentiates inflammation.

In this context, the inventors have identified Akkermansia muciniphila (A. muciniphila) as beneficial bacteria to counteract diet-induced obesity and metabolic disorders including low-grade inflammation (Everard A et al., Proc Natl Acad Sci USA, 2013). Therefore, the potential impact of A. muciniphila administration on the reward system in obese animals was evaluated.

### Materials and Methods

Operant wall test and statistical analysis: see example 1.

Mice experimental design: A cohort of 9-week-old specific-opportunistic and pathogen-free (SOPF) male C57BL/6J mice (Janvier laboratories, France) were housed in a controlled environment (room temperature of 22±2° C., 12 h daylight cycle) in groups of two mice per cage, with free access to sterile food (irradiated) and sterile water. Upon delivery, mice underwent an acclimatization period of one week, during which they were fed a control diet (CT, AlN93Mi, Research Diet, New Brunswick, NJ, USA). Mice were then randomly divided in two groups (20 mice, n=10/group), and fed for 8 weeks with a high-fat diet (HFD, 60% fat and 20% carbohydrates (kcal/100 g) D12492i, Research diet, New Brunswick, NJ, USA). One group was treated with Akkermansia muciniphila by oral gavage at a dose of 2.108 CFU suspended in sterile anaerobic PBS, as previously described (Everard A et al, Proc Natl Acad Sci USA, 2013). Placebo group was orally administered an equivalent volume of sterile anaerobic PBS containing a similar end concentration of glycerol (2.5% vol/vol). Treatments continued until the end of the experiment 8 weeks in total. After 4 weeks of follow-up, the mice entered the behavioral cages to perform the operant wall test. During this last test, mice were food-restricted and body weights were maintained at 85% of the initial body weight (before the behavioral tests), as previously described (de Wouters d'Oplinter A al, Gut Microbes, 2021). The caloric restriction allowed to potentiate the reward response to the stimuli.

### Results

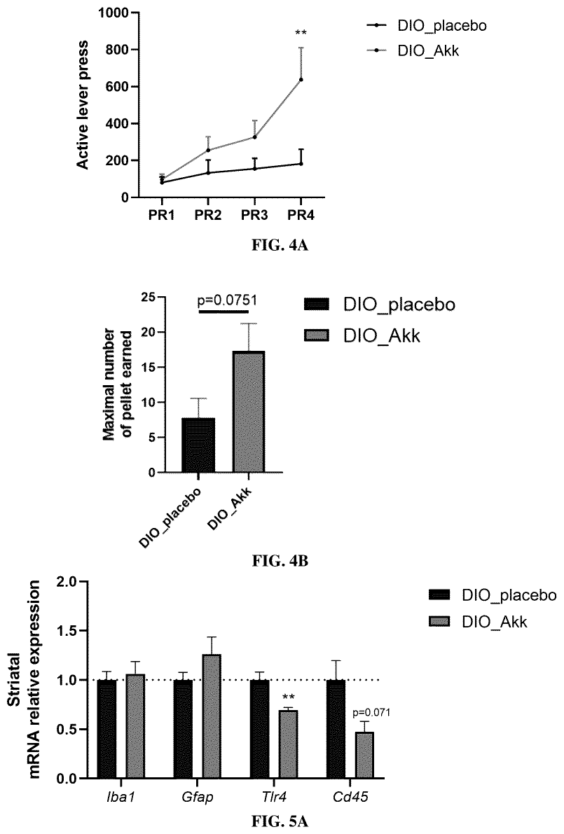

The food reward system was interrogated by investigating the motivation with an operant conditioning task. The progressive ratio sessions (PR) requiring an increasing number of lever presses to obtain a new food reward showed a statistically significant increase in the motivation of the obese mice treated with A. muciniphila at PR4 compared to obese mice treated with placebo (p=0.0022 for the comparison between DIO_placebo and DIO_Akk groups, FIG. 4A-B). A. muciniphila treated mice even reached a similar number of lever presses as lean mice during their first session of the progressive ratio (see FIG. 1B, mean=666 in Lean vs FIG. 3A, mean=638 in DIO_Akk). Consistent with the lever pressing parameter, the maximum number of reinforcers earned tended to be increased in obese mice treated with A. muciniphila compared to placebo treated mice. (p=0.075, FIG. 4B).

Considering the greater number of lever press to earn more sucrose pellets than obese placebo mice, these results show that the administration of A. muciniphila is able to restore the alteration of the motivation induced by a high-fat diet.

### Example 4: Beneficial Effects of Akkermansia muciniphila on the Reward System Imply a Reduction in Systemic and Neuroinflammation as Well as a Reduction in Striatal Lpl Expression

From a mechanistic perspective, the dysregulation of the reward system associated with obesity might be due to inflammation in reward-related brain areas. Based on the ability of A. muciniphila to reverse the motivational alterations induced by a high-fat diet (FIG. 4A-B) and because systemic anti-inflammatory effects have previously been described for this bacterium, the inflammation in reward-related brain area of obese mice treated or not with A. muciniphila was evaluated.

### Materials and Methods

Mice experimental design: see example 3.

RNA preparation, real-time qPCR and statistical analysis: see example 1.

Plasma multiplex analysis: Plasma levels of TNFa was measured by multiplex assay kits based on chemiluminescence detection and following manufacturer's instructions (Meso Scale Discovery, Gaithersburg, MD). Analyses were done using a QuickPlex SQ 120 instrument (MSD) and DISCOVERY WORKBENCH®4.0 software.

### Results

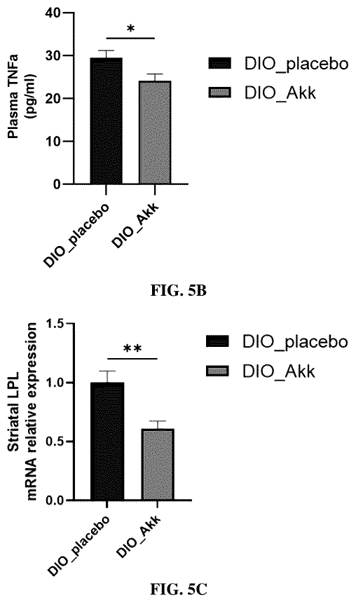

We found that the administration of A. muciniphila decreased plasma levels of TNFa (p=0.040 for the comparison between DIO_placebo and DIO_Akk mice, FIG. 5B). Consistent with this decrease in the level of a systemic inflammatory marker, the expression of Tlr4 was substantially decreased in the striatum of A. muciniphila-treated mice compared to placebo-treated mice (p=0.0015 DIO_placebo vs. DIO_Akk, FIG. 5A). The striatal expression of markers of infiltrating immune cells (Cd45) also tended to decrease in A. muciniphila-treated mice compared to placebo-treated mice (p=0.072 DIO_placebo vs. DIO_Akk, FIG. 5A). However, the striatal expression of markers of microglia (Iba1) and astrocytes (Gfap) was not changed between the DIO group compared to DIO_Akk group (FIG. 5A). Taken together, our results suggest thatA. muciniphila supplementation reverses motivational alterations associated with obesity, potentially through the modulation of Tlr4 expression and the infiltration of immune cells in the mesocorticolimbic structures.

According to previous studies, a potential mechanism linking alterations in behavioral reward and dopaminergic transmission involves central lipid sensing through the lipid-processing LPL. A similar increase in motivational performance for food-seeking behavior has been assessed with viral-mediated knockdown of LPL in mice in a progressive ratio operant conditioning paradigm (Berland C et al., Cell Metab, 2020; Cansell C et al, Mol Psychiatry, 2014). Importantly, in the cohort, mice receiving A. muciniphila showed a highly significant decrease in Lpl expression in the striatum compared to placebo-treated obese mice (p=0.0058, FIG. 5C).

muciniphila supplementation represents an additional and innovative approach to restore food reward behavior during obesity. These results open the way for further application of A. muciniphila in behavior improvement in other neuropsychiatric disorders such as Parkinson disease or Alzheimer disease, associated with CNS inflammation.

### Example 5: The Administration of Pasteurized Akkermansia muciniphila Restores the Hedonic/Liking and Tends to Improve the Motivational Components of Food Reward that are Altered by DIO

### Materials and Methods

Food preference test, operant wall test and statistical analysis: see example 1.

Mice experimental design: A cohort of 9-week-old specific-opportunistic and pathogen-free (SOPF) male C57BL/6J mice (Janvier laboratories, France) were housed in a controlled environment (room temperature of 22±2° C., 12 h daylight cycle) in groups of two mice per cage, with free access to sterile food (irradiated) and sterile water. Upon delivery, mice underwent an acclimatization period of one week, during which they were fed a control diet (CT, AlN93Mi, Research Diet, New Brunswick, NJ, USA). Mice were then randomly divided in two groups (32 mice, n=16/group) and fed with a high-fat diet (HFD, 60% fat and 20% carbohydrates (kcal/100 g) D12492i, Research diet, New Brunswick, NJ, USA) for 8 weeks. One group of HFD was treated with pasteurized Akkermansia muciniphila by oral gavage at a dose of 2×10⁸ cells suspended in sterile anaerobic PBS, as previously described (DIO_Akkpast) (pasteurization 30 min at 70° C.) (Plovier et al., Nat Med, 2017). The last group of HFD mice (DIO_Placebo) was orally administered an equivalent volume of sterile anaerobic PBS containing a similar end concentration of glycerol (2.5% vol/vol). Treatments continued until the end of the experiment 8 weeks in total. After 3 weeks of follow-up, the mice entered the behavioral cages to perform the food preference and operant wall tests. During this last test, mice were food-restricted and body weights were maintained at 85% of the initial body weight (before this behavioral test), as previously described (de Wouters d'Oplinter et al, Gut Microbes, 2021). The caloric restriction allowed to potentiate the reward response to the stimuli.

### Results

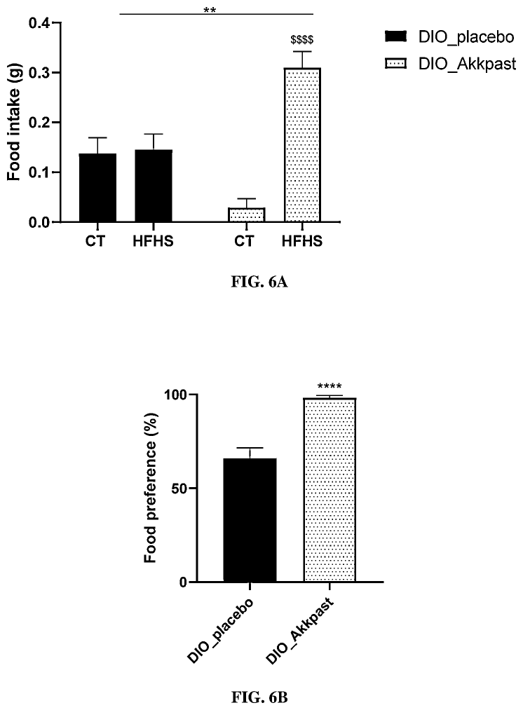

In this study, the food preference test to assess the liking component of the food reward was first performed. As expected, DIO_placebo mice do not show any tropism for palatable (HFHS) as they eat the same amount of HFHS diet than control diet, confirming the alteration of the liking component associated with obesity (FIG. 6). Importantly, results show that the administration of pasteurized A. muciniphila increases the hedonic food intake as pasteurized A. muciniphila supplemented mice (DIO_Akkpast) eat more HFHS than placebo treated mice (DIO_Placebo) (FIG. 6A). Moreover, pasteurizedA. muciniphila supplementation restores the preference for HFHS diet over CT diet (FIG. 6B). These results show that the supplementation in pasteurized A. muciniphila has beneficial effects on the liking component of food intake in the context of obesity, as it counteracts the high-fat diet-induced alterations. Therefore, pasteurized A. muciniphila restores liking component of food reward.

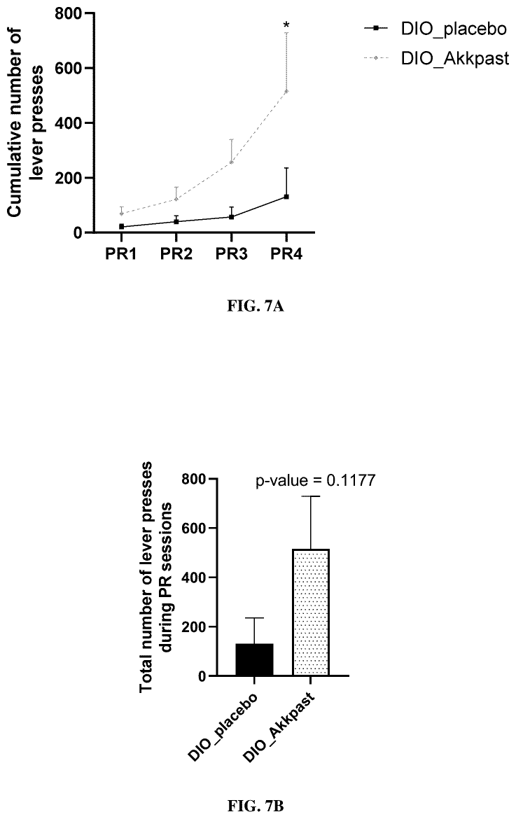

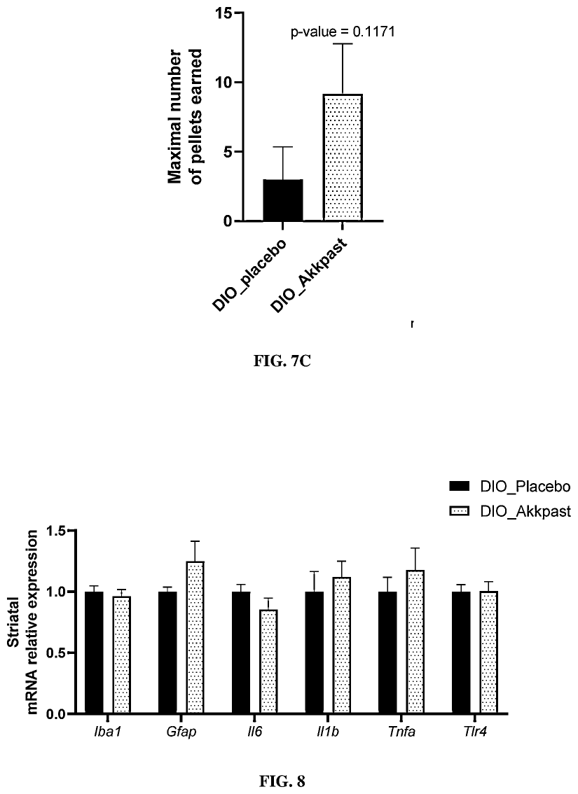

The motivation of the mice for a food reward was also assessed by the operant wall test. Interestingly, mice treated with pasteurized A. muciniphila (DIO_Akkpast) press more on the active lever than placebo-treated mice (DIO) (FIG. 7A-B).

Consistent with the lever pressing parameter, the maximum number of reinforcers earned tends to be increased in obese mice treated with pasteurized A. muciniphila compared to placebo treated mice (FIG. 7C). Therefore, pasteurized A. muciniphila improves wanting component of food reward.

Altogether, the study clearly shows beneficial effects of pasteurized A. muciniphila on the behavioral patterns of the food reward system.

### Example 6: Pasteurized Akkermansia muciniphila Modulates Inflammatory Profile of Microglial Cells in the Stratum During Obesity

### Materials and Methods

Mice experimental design: see example 5.

RNA preparation, real-time qPCR, immunofluorescence and statistical analysis: see example 1.

### Results

Next, the action of pasteurized A. muciniphila on inflammation and Lpl expression in reward-related brain areas was investigated. The expression of inflammatory markers in the striatum of HFD and HFD mice treated with pasteurized A. muciniphila was first assessed. When pasteurized, it seems that A. muciniphila does not impact the proinflammatory markers in reward-related brain area, since there was any significant difference between placebo and A. muciniphila-supplemented mice in the expression of Iba1 (microglia activation); Gfap (astrocytes); I16, Il1b and Tnfa (pro-inflammatory cytokines); and Tlr4 (receptor for LPS and some fatty acids) (FIG. 8).

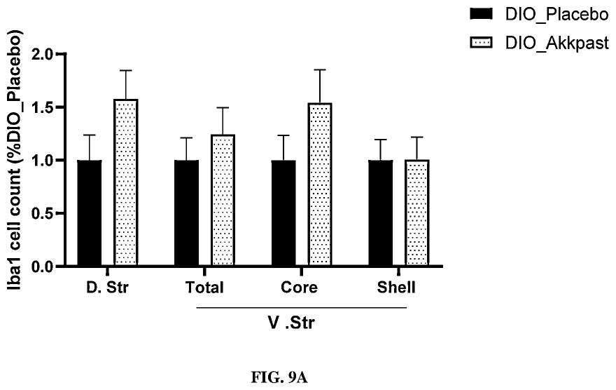

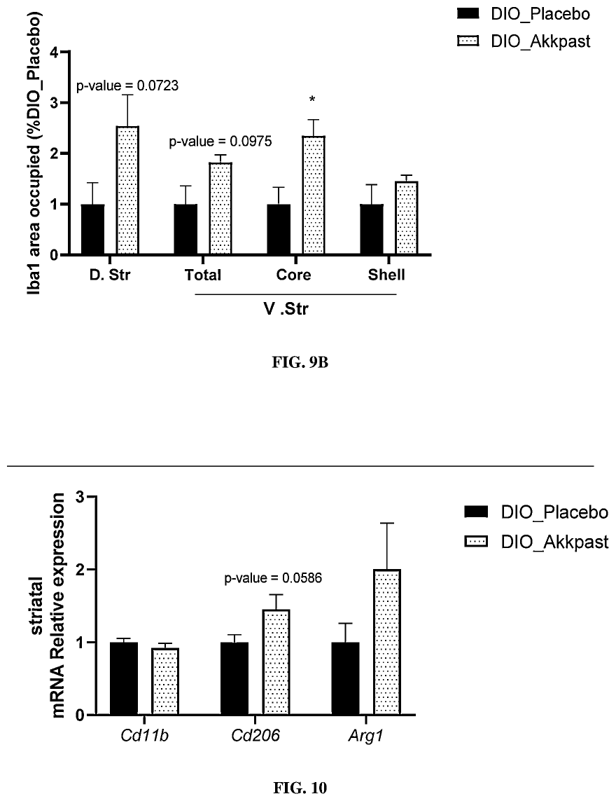

To deeper investigate and visualize inflammation, immunohistochemichal staining for microglial cells (immune cells in the brain) markers (Iba1) in reward-related area was performed. Surprisingly, it was found that the administration of pasteurized A. muciniphila induced an increase in the number of microglial cells in the dorsal striatum and ventral striatum core, as shown by the increase staining of Iba1+ cells in the DIO_Akkpast group compared to the lean group (FIG. 9A). Consistently, as the number of microglial cells is increased in these reward-related brain regions, an increase in the Iba1 area occupied in these regions in the DIO_Akkpast group was also observed (FIG. 9B).

Importantly, the activation of microglial cells (by the increase of number or size) can induce a pro- or anti-inflammatory phenotype. As Erny et al. has proved that the gut microbiota is able to regulate the microglia maturation and function, the effects of pasteurized A. muciniphila on the activation of anti-inflammatory microglial phenotype was further investigated (Erny et al, Nat Neurosci, 2015). The clusters of differentiation 11b and 206 (Cd11b and Cd206) and the arginase 1 (Arg1) are often used as markers of this anti-inflammatory microglial phenotype (Rossi et al, Cell Death Dis, 2018).

Interestingly, it was found a trend for an increase in two different anti-inflammatory markers Cd206 and Arg1 in the striatum of DIO_Akkpast compared to DIO_placebo group (FIG. 10). These data suggest that the increase and activation of microglial cells by pasteurized A. muciniphila are related to anti-inflammatory profile.

### Example 7: Beneficial Effects of Akkermansia muciniphila on the Reward System Imply a Reduction in Striatal Lpl Expression

### Materials and Methods

Mice experimental design: see example 5.

RNA preparation, real-time qPCR and statistical analysis: see example 1

### Results

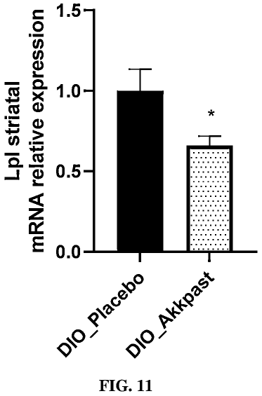

Mice receiving pasteurized A. muciniphila showed a significant decrease in Lpl expression in the striatum compared to placebo-treated obese mice (p=0.0456 after Student t-test between DIO_Placebo and DIO_Akk_Past, FIG. 11).

### REFERENCES

Avena N. M. and Bocarsly M. E., Dysregulation of Brain Reward Systems in Eating Disorders: Neurochemical Information from Animal Models of Binge Eating, Bulimia Nervosa, and Anorexia Nervosa. Neuropharmacology 2012; 63(1): 87-96.
Berland C, Cansell C, Hnasko T S, Magnan C, Luquet S. Dietary triglycerides as signaling molecules that influence reward and motivation. Curr Opin Behav Sci 2016; 9:126-35.
Berland C, Montalban E, Perrin E, Di Miceli M, Nakamura Y, Martinat M, et al. Circulating Triglycerides Gate Dopamine-Associated Behaviors through DRD2-Expressing Neurons. Cell Metab 2020; 31:773-90 e11.
Cani P D, Amar J, Iglesias M A, Poggi M, Knauf C, Bastelica D, et al. Metabolic endotoxemia initiates obesity and insulin resistance. Diabetes 2007; 56:1761-72.
Cani P D, Van Hul M, Lefort C, Depommier C, Rastelli M, Everard A. Microbial regulation of organismal energy homeostasis. Nat Metab 2019; 1:34-46.
Cansell C, Castel J, Denis R G, Rouch C, Delbes A S, Martinez S, et al. Dietary triglycerides act on mesolimbic structures to regulate the rewarding and motivational aspects of feeding. Mol Psychiatry 2014; 19:1095-105
Carlin J, Hill-Smith T E, Lucki I, Reyes T M. Reversal of dopamine system dysfunction in response to high-fat diet. Obesity (Silver Spring) 2013; 21:2513-21.
De Wouters d'Oplinter A, Rastelli M, Van Hul M, Delzenne N M, Cani P D, Everard A. Gut microbes participate in food preference alterations during obesity. Gut Microbes 2021; 13:1959242.
Decarie-Spain L, Hryhorczuk C, Lau D, Jacob-Brassard E, Fisette A, Fulton S. Prolonged saturated, but not monounsaturated, high-fat feeding provokes anxiodepressive-like behaviors in female mice despite similar metabolic consequences. Brain Behav Immun Health 2021; 16:100324.
Delbes A S, Castel J, Denis R G P, Morel C, Quinones M, Everard A, et al. Prebiotics Supplementation Impact on the Reinforcing and Motivational Aspect of Feeding. Front Endocrinol (Lausanne) 2018; 9:273.
Delzenne N M, Neyrinck A M, Bäckhed F, Cani P D. Targeting gut microbiota in obesity: effects of prebiotics and probiotics. Nature Reviews Endocrinology 2011; 7:639-46.
Depommier, C., et al., Pasteurized Akkermansia muciniphila increases whole-body energy expenditure and fecal energy excretion in diet-induced obese mice. Gut Microbes, 2020. 11(5): p. 1231-1245. Depommier C, Everard A, Druart C, Plovier H, Van Hul M, Vieira-Silva S, et al. Supplementation with Akkermansia muciniphila in overweight and obese human volunteers: a proof-of-concept exploratory study. Nat Med 2019; 25:1096-103.
Erny, D., et al., Host microbiota constantly control maturation and function of microglia in the CNS. Nat Neurosci, 2015. 18(7): p. 965-77.
Everard A, Belzer C, Geurts L, Ouwerkerk J P, Druart C, Bindels L B, et al. Cross-talk between Akkermansia muciniphila and intestinal epithelium controls diet-induced obesity. Proc Natl Acad Sci USA 2013; 110:9066-71.
Everard A, Plovier H, Rastelli M, Van Hul M, de Wouters d'Oplinter A, Geurts L, et al. Intestinal epithelial N-acylphosphatidylethanolamine phospholipase D links dietary fat to metabolic adaptations in obesity and steatosis. Nat Commun 2019; 10:457.
Frank G., Altered Brain Reward Circuits in Eating Disorders: Chicken or Egg? Curr Psychiatry Rep 2013; 15:396.
Guillemot-Legris O, Muccioli G G. Obesity-Induced Neuroinflammation: Beyond the Hypothalamus. Trends Neurosci 2017; 40:237-53.
Karcher N, Nigro E, Punčochář M, Blanco-Míguez A, Ciciani M, Manghi P, Zolfo M, Cumbo F, Manara S, Golzato D, Cereseto A, Arumugam M, Bui T P N, Tytgat H L P, Valles-Colomer M, de Vos W M, Segata N. Genomic diversity and ecology of human-associated Akkermansia species in the gut microbiome revealed by extensive metagenomic assembly. Genome Biol. 2021 Jul. 14; 22(1): 209.
Plovier H, Everard A, Druart C, Depommier C, Van Hul M, Geurts L, et al. A purified membrane protein from Akkermansia muciniphila or the pasteurized bacterium improves metabolism in obese and diabetic mice. Nat Med 2017; 23:107-13.
Rogers P. J. Food and drug addictions: Similarities and differences. Pharmacology, Biochemistry and Behavior 2017; 153:182-190.
Rossi, C., et al., Interleukin 4 modulates microglia homeostasis and attenuates the early slowly progressive phase of amyotrophic lateral sclerosis. Cell Death Dis, 2018. 9(2): p. 250.
van de Wouw M, Schellekens H, Dinan T G, Cryan J F. Microbiota-Gut-Brain Axis: Modulator of Host Metabolism and Appetite. J Nutr 2017; 147:727-45.
Volkow N D, Wang G J, Fowler J S, Telang F. Overlapping neuronal circuits in addiction and obesity: evidence of systems pathology. Philos Trans R Soc Lond B Biol Sci 2008; 363:3191-200.
Volkow N D, Wang G J, Fowler J S, Tomasi D, Baler R. Food and Drug Reward: Overlapping circuits in human obesity and addiction. Curr Topics Behav Neurosci 2012; 11:1-24.
Wang G J, Volkow N D, Logan J, Pappas N R, Wong C T, Zhu W, et al. Brain dopamine and obesity. Lancet 2001; 357:354-7.
Zhao J, Bi W, Xiao S, Lan X, Cheng X, Zhang J, et al. Neuroinflammation induced by lipopolysaccharide causes cognitive impairment in mice. Sci Rep 2019; 9:5790.

1-15. (canceled)

16. A method of preventing and/or treating reward dysregulation disorders; comprising administering, to a subject in need thereof, a composition comprising one or more bacteria from the genus Akkermansia and/or extracts and/or fragments thereof.

17. The method according to claim 16, wherein said bacterium is Akkermansia muciniphila or Akkermansia spp. and combinations thereof.

18. The method according to claim 16, wherein said reward dysregulation disorder is selected from a group consisting of mental disorders, neurological disorders, disorders due to side effects of a treatment and combinations thereof.

19. The method according to claim 18, wherein said reward dysregulation disorder is a mental disorder selected from a group consisting of addiction-related disorder, eating-related disorder, affective disorders, obsessive compulsive disorders, schizophrenia, attention deficit hyperactivity disorders (ADHD), autism spectrum disorder, anxiety disorder, and the like.

20. The method according to claim 19, wherein said reward dysregulation disorder is an addiction-related disorder selected from a group consisting of alcohol-related addiction, drug-related addiction, game-related addiction, and the like.

21. The method according to claim 18, wherein said reward dysregulation disorder is a neurological disorder selected from a group consisting of Parkinson's disease, Tourette Syndrome, and the like.

22. The method according to claim 18, wherein said reward dysregulation disorder is a disorder due to side effects of a treatment is selected from a group consisting of game-addiction, shopping-addiction, eating-addiction such as hyperphagia, hypersexuality, and the like.

23. The method according to claim 19, wherein said reward dysregulation disorder is an eating disorder is selected from a group consisting of bulimia nervosa, binge eating disorder, anorexia nervosa including restricting type and Binge-eating/purging type, pica, rumination disorder, purging disorder, night eating syndrome, avoidant restrictive food intake disorder, overweight-related disorder and obesity-related disorders, food addiction, eating addiction, food craving, food seeking, compulsive eating disorders, impulsive eating disorders, unsuccessful caloric restriction diet, non-responders to weight loss or non-responding to dietary intervention for losing weight, and the like.

24. The method according to claim 16, wherein said composition further comprises one or more active agent(s).

25. The method according to claim 24, wherein said active agent is a therapeutic agent or a nutritional agent.

26. The method according to claim 24, wherein said active agent is a beneficial microbe selected from a group consisting of bacteria from the family Verrucomicrobia, from the family Tannerellaceae, from the family Clostridiaceae, from the family Peptostreptococcaceae, from the family Prevotellaceae, from the family Methylobacteriaceae, from the genus Parabacteroides, from the genus Turicibacter, from the genus Coprococcus, from the genus Knoellia, from the genus Prevotella, from the genus Staphylococcus, and the like.

27. The method according to claim 16, wherein said composition is in the form of a pharmaceutical composition further comprising a pharmaceutically acceptable carrier.

28. The method according to claim 16, wherein said composition is in the form of a nutritional composition further comprising a nutritionally acceptable carrier.

29. A method for adjuvanting a treatment administered to a subject suffering from a reward dysregulation disorder, comprising administering, to a subject in need thereof, a composition comprising one or more bacteria from the genus Akkermansia and/or extracts and/or fragments thereof.

30. The method according to claim 16, wherein said composition is comprised in a kit, which further comprises means to administer said composition.

31. The method according to claim 29, wherein said composition is comprised in a kit, which further comprises means to administer said composition.
